(19) United States
(12) Patent Application Publication
(10) Pub. No.: US 2008/0193377 A1
(43) Pub. Date: Aug. 14, 2008

(54) Radiolabeled Nanohybrids Targeting Solid Tumor Neovasculature and Method of Using Same

(75) Inventors: Bruce R. Line, Cockeysville, MD (US); Beth Line, Cockeysville, MD (US); Hamidreza Ghandehari, Salt Lake City, UT (US); Sergey Baklanov, Indianapolis, IN (US)

Correspondence Address: FOLEY AND LARDNER LLP;SUITE 500, 3000 K STREET NW, WASHINGTON, DC, 20007, US

(73) Assignee: University of Maryland, Baltimore

(21) Appl. No.: 11/571,340

(22) PCT Filed: Jun. 28, 2005
(86) PCT No.: PCT/US05/22666

§ 371 Date: Jan. 8, 2008

Related U.S. Application Data
(60) Provisional application No. 60/582,818, filed on Jun. 28, 2004.
(60) Provisional application No. 60/667,945, filed on Apr. 4, 2005.

(51) Int. Cl. A61K 51/06; C07K 7/64; C07F 19/00; A61K 51/00; C07C 233/09; C07F 13/00

(52) U.S. Cl. 424/1.65; 530/317; 534/10; 564/204; 534/14; 424/1.11

(57) ABSTRACT

Nanohybrid polymer conjugates that provide a “platform” delivery system is disclosed. The delivery platform provides a multi-focused therapeutic regimen that may be tailored to combat a host of cancers, including advanced-stage, therapy-resistant tumors. The nanohybrids of the instant invention incorporate a configurable polymeric backbone, are multivalent (e.g., may incorporate several targeting ligands), and have the capacity to carry multiple classes of “payloads” (e.g., alpha-, beta-, gamma- and positron-emitting isotopes). The polymer conjugates comprise a single molecular species that can be useful not only in diagnostic assessment, but also in tailoring therapies to suit a variety of cancers.

### CLAIM OF PRIORITY

This application claims priority to U.S. application Nos. 60/582,818 and 60/667,945, filed Jun. 28, 2004 and Apr. 4, 2005, respectively, the disclosures of which are hereby incorporated in their entirety by reference.

### GOVERNMENT INTEREST

The invention described herein was made with Government support by the National Institutes of Health under grant numbers R21CA81492-01A1 and R21CA91770A, the Department of Defense under grant number DAMD170010004, and congressional allocation through the American Russian Cancer Alliance (1 R06SP04146-001-00). The United States Government has certain rights in the invention.

### FIELD OF THE INVENTION

The present invention relates generally to cancer therapy. More specifically, the present invention relates to the products and methods of treating solid tumors with polymeric conjugates, wherein the polymeric conjugates specifically target endothelial cells supporting tumor angiogenesis.

### BACKGROUND OF THE INVENTION

Significant progress has been made in the treatment of the advanced solid tumors including breast, colon, lung and prostate. Randomized trials have demonstrated improvements in terms of response, median survival, and overall survival with the advent of new chemotherapy agents. However, despite these advances, long term survival, i.e. survival beyond 3 years, is relatively unusual, and there are few long term (i.e., >5 years) survivors. Hence, there is a significant need for the treatment of these malignancies.

Targeting the endothelial cells supporting tumor angiogenesis provides an alternative method for cancer diagnosis and therapy. For one, angiogenesis is both essential for tumor growth beyond 1-2 mm size and is highly specific for neoplasia. For example, except in wound healing or in the female reproductive tissues, only 0.01% of normal endothelial cells are actively involved in angiogenesis. Thus, a therapy directed to tumor vessels generally is expected to have broader applicability over any tumor-specific treatment.

Furthermore, studies examining microvessel density reveal a significant statistical correlation between microvessel density and clinical stage, histopathological stage, and disease-specific survival for many types of cancer. Microvascular density is found to have independent prognostic significance when compared with traditional prognostic markers by multivariate analysis in prostate cancer, malignant melanomas, multiple myeloma, central nervous system tumors, and carcinomas of the breast, lung, head and neck, nasopharynx, gastrointestinal tract, bladder, endometrium, ovaries, testes, and reproductive tract.

Bevacuzimab (Avastin), an anti-vascularization monoclonal antibody has recently been approved for the treatment of colorectal cancer based upon a randomized controlled trial documenting survival advantage. Strategies targeting mediators of neovascularization, while rational, have several significant drawbacks. For example, there is often redundancy in angiogenic pathways and targeting a single molecule is unlikely to be of long term benefit. A more logical approach may be to target the neovasculature itself with molecularly directed radiotherapy.

Encouraging results have been achieved with radioimmunotherapy in hematological malignancies. Monoclonal antibodies, for example, have been used to target beta radiotherapy (e.g., Zevalin and Bexaar) to successfully treat lymphomas and other B-cell malignancies. However, there has been less success in treating solid tumors with targeted radiotherapy. Within the bounds of acceptable bone marrow toxicity, the typical accretion of 0.001-0.01% injected dose (ID %) of the radiolabeled antibody/gram of tumor produces less than a third of the typical >50 Gy needed to achieve therapeutic responses. The low tumor accretion of targeted radioactivity likely results from a number of complex factors, including the fact that tumor cells that lie outside the bloodstream are poorly accessible to many targeting molecules that are delivered via same.

The endothelial location of angiogenesis related integrins may provide a critical advantage over other tumor related targets because a radiopharmaceutical does not need to diffuse into the extravascular space. Accordingly, there is a need for radiopharmaceutical products and methods for treating vascularized solid tumors.

### SUMMARY OF THE INVENTION

The foregoing needs are met, to an extent, by the present invention, wherein in one embodiment an anti-angiogenic polymer conjugate (APC) for treatment of solid tumors is provided comprising: a polymer backbone capable of modification with a plurality of side chains, at least one side chain comprising a chemical moiety targeting cell-surface proteins of endothelial cells at an angiogenic site. The cell surface proteins may be on the luminal surface, the cell-surface proteins may be an integrin, and the integrin is may be αᵥβ₃ integrin. In some embodiments, the chemical moiety may be a ligand for a cell-surface receptor, such as, for example, an integrin. The integrin may be αᵥβ₃ integrin, and the ligand may be RGD4C or RGDfK. The ligand content may comprise less than about 50 mole percent of the polymer conjugate. The polymer conjugate may further comprise at least one side chain comprising a chelator capable of chelating a pharmaceutically acceptable radioactive label.

In another embodiment of the present invention, an anti-angiogenic polymer conjugate (APC) for treatment of solid tumors is provided, comprising: a polymer backbone capable of modification with a plurality of side chains, at least one side chain comprising a chelator, said chelator harboring a pharmaceutically acceptable alpha emitting radioactive label. The alpha emitting radioisotope may be ²¹³Bi or ²¹⁰Po. The polymer conjugate may further comprise at least one side chain comprising a chemical moiety targeting cell-surface proteins of endothelial cells at an angiogenic site.

In yet another embodiment of the invention, an anti-angiogenic polymer conjugate (APC) of less than about 45 kD for treatment of solid tumors, is provided comprising: a polymer backbone capable of modification with a plurality of side chains, at least one of the side chains comprising a chemical moiety targeting cell-surface proteins of endothelial cells at an angiogenic site, and at least one of the side chains comprising a chelator capable of chelating a pharmaceutically acceptable radioactive label. In some embodiments, the polymer backbone may be water soluble and/or electronegative. The polymer backbone may also be N-(2-hydroxypropyl) methacrylamide (HMPA). At least one of the plurality of side chains can comprise a glycylglycine moiety, and one of the side chains may also comprise COOH groups. The COOH groups comprise less than about 50 mole percent, and more preferably, less than about 40 mole percent of the polymer conjugate in other embodiments. The radioactive label may be an alpha, beta, gamma, or positron emitting radioisotope. In some case, the beta emitting radioisotope is ⁹⁰Y, ¹³¹I, ¹⁸⁸Re, ¹⁸⁶Re or ¹⁷⁷Lu; or the radiolabel may be selected from the group consisting of ¹²⁴I and ^{99m}Tc. The chelator may be selected from the group consisting of dipyridyllysine (“DPK”), m-hydroxybenzoic acid (“HBA”), and 1,4,7,10-tetraazacyclododecane-1,4,7,10-tetraacetic acid (“DOTA”). The DOTA content comprises from about 5 mole percent to about 10 mole percent of the polymer conjugate; the chelator content comprises from about 5 mole percent to about 40 mole percent of the polymer conjugate; and chelator content comprises from about 10 mole percent to about 30 mole percent of the polymer conjugate in some embodiments.

In further still another embodiment of the present invention, a method of radiotherapy for the treatment of solid tumors is provided, comprising: administering to a mammal harboring a solid tumor in need of said treatment, an effective dose of an anti-angiogenic polymer conjugate (APC) of less than about 45 kD, comprising: a polymer backbone capable of modification with a plurality of side chains, at least one of the side chains comprising a chemical moiety targeting cell-surface proteins of endothelial cells at an angiogenic site, and at least one of the side chains comprising a chelator capable of chelating a pharmaceutically acceptable radioactive label, wherein the chemical moiety is directly coupled to the polymer backbone with a chemical spacer.

In further still yet, another embodiment, a method of localizing a radioactive nucleotide at the site of a solid tumor in a mammal is provided, comprising: a polymer backbone capable of modification with a plurality of side chains, at least one of the side chains comprising a chemical moiety targeting cell-surface proteins of endothelial cells at an angiogenic site, and at least one of the side chains comprising a chelator capable of chelating a pharmaceutically acceptable radioactive label, wherein the chemical moiety is directly coupled to the polymer backbone with a chemical spacer.

In further yet another embodiment, a method of a determining a suitable radiotherapeutic regimen for treatment of a vascularized solid tumor in a mammal based on location and distribution of a tracer radioactive label is provided, comprising: (a) administering to the mammal a tracer dose of an APC according to claim 1; wherein the pharmaceutically acceptable radioactive label is a tracer label; (b) determining the location and concentration of the tracer radioactive label within said mammal; (c) calculating an amount of radioactivity required to deliver a therapeutic dose of a pharmaceutically acceptable radioactive label which is therapeutic. The method may have a tracer label that is ¹²⁴I. The determination step (b) may be performed by one or a combination of positron-emission tomography and computerized tomography, or the determination step (b) may be performed over a predetermined period of time, or the determination (b) may further comprise modeling the kinetics of radioactivity in the tumor during the predetermined period of time. The method may further comprise administering to the mammal a dose of an APC according to claim 1 based on the amount calculated in step (c), wherein pharmaceutically acceptable radioactive label is therapeutic radioactive label.

In still yet another embodiment of the present invention, a method of determining a suitable radiotherapeutic regimen for treatment of a vascularized solid tumor in a mammal based on location and distribution of a tracer radioactive label is provided, comprising: (a) administering to the mammal a tracer dose of an APC according to claim 1; wherein the pharmaceutically acceptable radioactive label is a tracer label; (b) determining the location and concentration of the tracer radioactive label within said mammal; (c) calculating an amount of radioactivity required to deliver a therapeutic dose of a pharmaceutically acceptable therapeutic radioactive label.

There has thus been outlined, rather broadly, certain embodiments of the invention in order that the detailed description thereof herein may be better understood, and in order that the present contribution to the art may be better appreciated. There are, of course, additional embodiments of the invention that may be described below and which may form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art may appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a schematic depicting how a delivery system in accordance with the invention may provide a versatile platform for (1) planning molecularly guided radiotherapy (e.g., imaging), (2) delivery of therapeutic effectors, and (3) following the response to treatment for a wide range of human cancers.

FIG. 2 shows three examples of nanohybrid architecture in accordance with the instant invention. All of the examples include the HPMA copolymer containing the reactive comonomer residue (MAGGONp) that subsequently is reacted with the RGD4C targeting ligand. (A) A nanohybrid that also contains Methacryloylglycylglycyl-carboxylate (MAGGCOOH) for electronegative charge, N-methacryloyltyrosinamide (MA-Tyr) for iodine coupling and Methacryloylglycylglycyldipyridyllysine (MAGGDPK) for ^{99m}Tc chelation. (B) Another nanohybrid in which the MAGGCOOH is replaced by either methacryloyl-glycylglycyl-p-aminobenzyl-1,4,7,10-tetraazacyclododecane-1,4,7,10-tetraacetic acid (MAGGDOTA) or APMA-CHX-A″-DTPA as shown. (C) This nanohybrid adds only APMA-maleimido-propane which is used to subsequently attach the thiol on CYS-HIS-CHX-A″-DTPA shown at the right of the panel. This design separates the backbone construction from the radioisotope chelation groups. (D) RGD4C, targeting peptide recognizable by αᵥβ₃. The control peptide RGE4C (E) differs by one carbon from RGD4C. (H) APMA-CHX-DPTA. The peptides were conjugated to MAGGONp side chains in polymer precursors by aminolysis. Unconjugated MAGGONp groups were hydrolyzed to generate COOH groups. Conjugates with DPK were radiolabeled with ^{99m}Tc for imaging and biodistribution studies.

FIG. 3 depicts results from an in vitro adhesion assay using human umbilical vein endothelial cells (HUVECs). The inhibition of adhesion of HUVECs onto fibrinogen-coated surfaces is mediated by specific recognition of RGD4C by αᵥβ₃ integrins on the surface of HUVECs and is indicated by decrease of optical density with increasing concentration. The values represent means of triplicate ±SD. Free RGE4C (biologically inert peptide), HPMA-RGE4C, and HPMA alone did not show any inhibition of binding (upper curves). Similar inhibition properties indicated that HPMA-RGD4C conjugate showed specific binding and was comparable to free RGD4C and Tc-99m radiolabeled HPMA-RGD4C indicating no loss of activity after conjugation (lower curves).

FIG. 4. shows residual radioactivity in percent injected dose per gram (% ID/g) of organ tissue 24 hours post-intravenous injection of ^{99m}Tc labeled copolymers. The tumor localization of HPMA copolymer-RGD4C conjugate (n=6, white bars) is significantly (p<0.001) greater than HPMA copolymer-RGE4C conjugate (n=6, black bars) and HPMA copolymer (n=5, gray bars). The excised organ data is expressed as mean ±SD.

FIG. 5 depicts results from an in vivo imaging and biodistribution assay of ^{99m}Tc labeled HPMA copolymer conjugates in mice bearing DU145 prostate tumor xenografts. Scintigraphic images, 70 min & 24 hours post injection of HPMA copolymer-RGD4C/RGE4C conjugates (400-500 μCi), using a gamma camera. 24 hours images showed higher tumor localization of the HPMA-RGD4C conjugate than the HPMA-RGE4C conjugate due to active RGD4C mediated targeting.

FIG. 6 shows the time-dependent organ distribution (% ID/g) 24 (n=6, horizontal hatch), 48 (n=5, white) and 72 hours (n=5, black) post-intravenous injection of ^{99m}Tc-HPMA copolymer-RGD4C conjugates. A time-dependent decrease of HPMA copolymer-RGD4C conjugate is noted in organs (blood, heart, lung, liver, spleen and kidney). The excised organ data is expressed as mean ±SD. *p<0.05 compared to 24 hours, **p<0.05 compared to 24 and 72 hours.

FIG. 7 depicts tumor/blood ratios of HPMA copolymer-RGD4C conjugate 24, 48 and 72 hours post-intravenous injection. The ratios increase significantly over time indicating rapid blood clearance and sustained tumor accumulation. The values are expressed as mean ±SD of at least five mice. *p<0.05 compared to 24 hours, **p<0.05 compared to 24 and 48 hours.

FIG. 8 depicts results from an in vivo 24 hr biodistribution assay comparing ^{99m}Tc labeled HPMA-peptide conjugates and free peptides in mice bearing DU145 prostate tumor xenografts. Data expressed as percentage injected dose per gram (% ID)/g). Significantly higher (p<0.001) tumor uptake of both HPMA-RGD4C and free RGD4C-DPK compared to controls HPMA-RGE4C and RGE4C-DPK. HPMA-RGD4C conjugate accumulated in the tumor more than RGD4C-DPK. HPMA-RGD4C also demonstrated significantly (p<0.001) less accumulation in liver and kidney than RGD4C-DPK. The data demonstrate that actively targeted polymeric conjugates increase tumor accumulation and decrease nonspecific uptake by other tissues.

FIG. 9 shows the residual radioactivity in % injected dose (% ID) per gram of organ tissue 24 hours after injection of neutral ^{99m}Tc-HPMA copolymer fractions (non-tumor bearing animals). The excised organs were counted using a gamma counter and data expressed as mean ±SD (number of animals/group is shown). This data clearly demonstrates that incorporation of negative charge could significantly enhance elimination of the HPMA copolymers from the body and reduce extravasation in normal tissues.

FIG. 10 shows the residual radioactivity in % injected dose (% ID) per gram of organ tissue 24 hours after injection of electronegative ^{99m}Tc-HPMA copolymer fractions (non-tumor bearing animals). The excised organs were counted using a gamma counter and data expressed as mean ±SD (number of animals/group is shown). This data clearly demonstrates that incorporation of negative charge could significantly enhance elimination of the HPMA copolymers from the body and reduce extravasation in normal tissues.

FIG. 11 shows the compartmental modeling (SAAMII Univ. Washington) of Bi-213 and Po-210 dose based on data derived from scintigraphic studies of the biodistribution of HPMA-RGD4C. Compartmental model and transfer constants were identical for this comparison of relative tissue dose with differences due solely to alpha radioisotope half-life. Administered activities in the models were chosen to deliver 100 Gy to tumor. There is a substantially higher blood dose for Bi-213 under the model assumptions.

FIG. 12 depicts residual radioactivity (% ID/g) 1, 24, 48 and 72 hours post-IV injection of ^{99m}Tc labeled HPMA copolymer-RGD4C conjugate in DU145 prostate tumor xenograft bearing SCID mice. *p<0.05 compared to 1 hour. **p<0.01 compared to 1 hour. Six animals all groups except 3 at 1 hour.

FIG. 13 shows images of two typical SCID mice bearing DU145 human prostate tumor xenografts 48 hours post-intravenous injection of ^{99m}Tc labeled RGD4C copolymer conjugate showing marked localization in tumor (right flank) with background activity in the kidneys and bladder.

FIG. 14 depicts the effect of ⁹⁰Y labeled HPMA copolymer-RGD4C conjugate treatment on DU145 growth in SCID mice. Animal groups treated with single dose of 100 mCi (p<0.03) and 250 mCi (p<0.01) ⁹⁰Y-HPMA-RGD4C conjugate showed significant tumor growth reduction as compared to the untreated controls by day 7 post-treatment. Data are presented as mean of tumor volume (cm3)±SD (n=6 mice per group).

FIG. 15 shows tumor samples from 250 mCi treatment showed cellular drop out consistent with increased apoptosis (black arrow). There were increased apoptotic bodies, eosinophilic bodies (thanatosomes, open arrow) and pronounced nuclear atypia indicative of treatment effect (hatched arrow).

FIG. 16 depicts the effect of ²¹⁰Po labeled HPMA copolymer-RGD4C conjugate treatment on DU145 growth in SCID mice. Animal groups treated with single dose of 5, 1, or 0.2 mCi ²¹⁰Po-HPMA-RGD4C conjugate showed significant tumor growth reduction as compared to the untreated controls by day 7 post-treatment. The 1 and 0.2 mCi treatment groups showed an initial depression of tumor size but subsequent recovery and regrowth, presumably due to non-treated pre-angiogenic zones in the tumor. Data are presented as mean of tumor volume (cm3)±SD (n=6 mice per group).

FIG. 17 shows calculated isodose distribution superimposed on a CT slice.

### DETAILED DESCRIPTION

“Nanohybrid” polymer conjugates of the instant invention provide a “platform” delivery system from which a multi-focused therapeutic regimen may be tailored to combat a host of cancers, including advanced-stage, therapy-resistant tumors. As will be described in greater detail below, the nanohybrids of the instant invention incorporate a configurable polymeric backbone, are multivalent (e.g., may incorporate several targeting ligands), and have the capacity to carry multiple classes of “payloads” (e.g., alpha-, beta-, gamma- and positron-emitting isotopes).

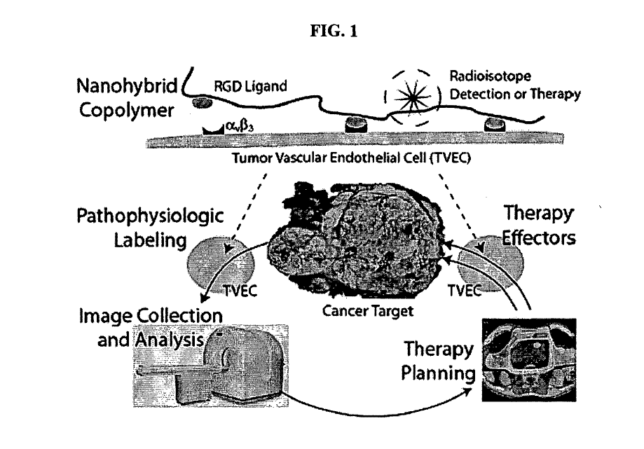

The polymer conjugates comprise a single molecular species that can be useful not only in diagnostic assessment, but also in tailoring therapies to suit a variety of cancers (FIG. 1). The facility to transport any of a number of targeting ligands and radioisotopic passengers in a single vehicle provides a heretofore unprecedented, highly flexible means to tailor image guided radiotherapy to individual patient needs. Furthermore, the peptide-polymer conjugate architecture provides a number of ways to control nanohybrid tumor binding strength, kidney clearance, and normal tissue biodistribution, to both enhance radiation delivery and reduce toxicity.

In other words, the design of the nanohybrid delivery system is robust enough that regardless of the therapeutic payload, the biodistribution of the delivery system remains independent of the payload. Thus, a physician is able to interchange therapeutic effectors without changing the design of the delivery system, which is a unique aspect not afforded by currently available methods. Further, selection of the delivery drug may be separated from the choice of a set of effectors appropriate to a given patient.

While the polymer conjugates of the instant invention can be specifically targeted to tissue-specific tumors, they are preferably targeted to markers common to tumors, generally, for broadest application. As well, without a specific targeting ligand, the polymer conjugates can accumulate passively through permeable blood vessels in tumor tissues by a process called enhanced permeability and retention (EPR). However, this passive localization is greatly improved by active targeting.

In accordance with one embodiment of the invention, the polymer conjugates are targeted to angiogenic tumor vessel endothelial cells (“TVEC”) and hereinafter referred to as anti-angiogenic polymer conjugates (“APCs”). Table 1 provides non-limiting examples of some receptors present on endothelial cell surfaces and some corresponding ligands thereto.

TABLE 1
Endothelial cell surface receptors and ligands thereto

| Receptors | Ligands |
|---|---|
| VEGF-R1 | VEGF-A, VEGF-B, PlGF |
| VEGF-R2 | VEGF-A, VEGF-B, VEGF-C, VEGF-D |
| VEGF-R3 | VEGF-C, VEGF-D |
| Tie-1 | Ang-1, Ang-2, Ang-3, Ang-4 |
| Ti-2 | Ang-1, Ang-2, Ang-3, Ang-4 |
| α1β1 | Collagen, laminin |
| α2β1 | Collagen, laminin |
| α3β1 | Laminin-5, thrombospondin |
| α4β1 | fibronectin |
| α5β1 | Fibronectin, fibrin |
| α6β1 | laminin |
| α8β1 | Fibronectin, |
| α9β1 | Tenascin-C |
| αVβ3 | Fibronectin, vitronectin, laminin, |
| | fibrinogen, thrombospondin |
| αVβ5 | Fibronectin |
| α6β4 | laminin |
| αVβ8 | vitronectin |
| Endoglin | TGFβ |
| Platelet derived growth | Platelet derived growth factor |
| factor receptor | |
| Thrombin receptor | thrombin |

Of the molecular markers associated with neovascular angiogenesis, the αᵥβ₃ integrin is selected as one of the target molecular markers associated with neovascular angiogenesis. αᵥβ₃ integrin is an endothelial cell surface receptor of vitronectin and is thought to be concentrated on the apical surface of forming or newly formed blood vessels, while absent or barely detectable in established blood vessels.

A number of peptide and peptidomimetic ligands that target sites associated with angiogenic vessels may be used as “homing” devices to direct therapy to the tumor bed in accordance with the teachings herein. In the case of αᵥβ₃, high affinity selective peptide ligands have been identified by screening phages. Phage bearing the cyclic nona-peptide, CDCRGDCFC (“RGD4C”), concentrate in tumors and bind selectively to αᵥβ₃ and αᵥβ₅ integrins. Thus, the early appearance of an angiogenic response in a tumor (relative to tumor growth) and the highly selective expression of αᵥ integrins in the neovascular tissue in a broad spectrum of tumors coupled with the critical role the angiogenic response plays in tumor growth and progression makes αᵥβ integrins a preferable target for both diagnostic imaging and directed therapy in some embodiments (FIG. 1).

The RG4DC peptide ligand is highly specific for the αᵥβ₃ integrin expressed on the luminal surface of angiogenic endothelial cells. This ligand can enable diagnostic and therapeutic strategies, when coupled to a polymeric backbone carrying an arsenal of radioactive isotopes. For example, images revealing disease stage and therapy effect can be produced by gamma and positron emitting isotopes. Meanwhile, alpha emitting isotopes that injure the angiogenic vascular bed may prove highly effective in inducing tumor necrosis as beta emitting isotopes provide direct tumor radiotherapy (FIG. 1).

In one embodiment, the RGD4C αᵥβ₃ ligand is used to target a polymeric backbone capable of carrying a versatile payload of diagnostic and therapeutic radioisotopes to the angiogenic vessel. As disclosed herein, the conjugation of the RGD4C peptide onto a polymer backbone can significantly enhance the tumor tissue uptake in comparison to the RGD4C peptide itself. In another embodiment, the RGDfK ligand is used to target a polymeric backbone capable of carrying a versatile payload of diagnostic and/or therapeutic effectors to an angiogenic vessel.

HPMA copolymers are one class of water-soluble synthetic polymeric carriers that have been extensively characterized as biocompatible, non-immunogenic and non-toxic. One advantage of HPMA copolymers over other water-soluble polymers is that they may be tailored through relatively simple chemical modifications to regulate their respective drug and targeting moiety content. The tailoring may be designed for biorecognition, internalization, or subcellular trafficking, depending on the specific therapeutic needs. Further, the molecular weight and charge of these copolymers may be manipulated to allow renal clearance and excretion from the body, or to alter biodistribution while allowing tumor targeting. Alternatively, these polymers can be designed to accumulate passively in tumor tissues by EPR effect.

Regarding “versatile payloads,” polymer conjugates of the instant invention may be constructed to bear one or several of radioactive isotopes including alpha, beta, gamma and positrons emitters. Radioisotopes have at least one significant advantage over other therapy agents, namely, the emission of energy that can kill at a distance from the point of radioisotope localization. This “diameter of effectiveness” may be the solution to overcome the problem of tumor heterogeneity. It should be noted that the polymer conjugates may be designed, and even preferably designed, with the capacity to chelate and deliver multiple isotopes whether or not all of that capacity is used in a given regimen. For example, an APC may be developed with the capacity to carry alpha, beta, gamma and positron isotopes, whereas it may be loaded with one, two or three of the isotopes.

Conjugates bearing an alpha or beta emitter can be designed to cause substantial, highly directed injury of the endothelial lining of vessels feeding a tumor. Alpha radiation delivered by vascular targeted nanohybrids can provide an efficient analogue to antivascular therapy and can also be effective in zones of hypoxia. These high energy, short-range (i.e., 50-80 μm) isotopes, (e.g., ²¹⁰Po) can destroy angiogenic vascular endothelial cells, compromise tumor blood flow, and effectively “starve” tumor cells. Vascular injury exposes the underlying thrombogenic submatrix, which triggers the formation of a hemostatic plug.

Alpha particles can also provide more effective radiotherapy in zones of low oxygen tension and kill tumor cells surrounding vessels, a feature common to tumors larger than about 5-10 mm and a significant cause of therapy failure by conventional approaches. Short range alpha particle radiation is highly lethal to neighboring cells, making it ideal for small or micrometastatic tumors. To avoid systemic toxicity, however, polymeric-peptide conjugates may be further tailored to reduce uptake in non-tumor capillary beds and control renal elimination of the complex from the blood.

Moderate range (i.e., 1-5 mm) beta particle emitting isotopes (e.g., ¹³¹I and ⁹⁰Y) may reduce the need to target every tumor cell and to kill cells that can reside in the pre-angiogenic peripheral tumor rim. Beta radiation has demonstrated substantial effectiveness in preclinical models where a sufficient therapeutic index was achieved due to the range of the beta particle and its cross fire effect.

Low energy, long range gamma emitting isotopes (e.g., ¹¹¹In and ¹²³I) can be employed to provide a diagnostic signal to detect the presence of occult tumor sites and monitor the effect of therapy. For example, gamma emitting isotopes may be used to detect cancer stage and evaluate the biologic aggressiveness of the cancer. Finally, the moderate energy positron emitting isotope, ¹²⁴I, can be employed to allow, for example, quantitative PET/CT imaging and pharmacokinetic analysis of tumor uptake of the polymer conjugates.

Using pre-treatment administration of a radiopharmaceutical radiolabeled with a diagnostic isotope, it may be possible to plan therapy dose levels by predicting the radiation absorbed dose to both tumors and surrounding healthy organs. This is most useful where quantitative, high-resolution imaging systems are coupled with dose-estimation software (FIG. 1). Further value can be provided where biological dose-response data is available to predict biological effect from radiation absorbed dose information.

For example, clinical practice using external beam radiation therapy is based on assessment of the radiation absorbed dose in both tumor and dose-limiting normal organs. The incorporation of new imaging methods, combined with patient-specific radiation transport simulations, may be used to develop treatment planning as the quantitative foundation for image guided radionuclide therapy. This information is critical to more accurate prediction of effective dose delivered by alpha and beta emitting isotopes. ¹²⁴I labeled nanohybrids can also provide the means to tailor therapy for individual patients through molecularly guided, internal source, radiation treatment planning.

Tumor architecture and micro environments are known to be heterogeneous and vary with tumor size. Thus, the quantity of angiogenesis related vascular target may vary by tumor histology, tumor size, and location within the tumor. For example, the effectiveness of radioimmunotherapy (RIT) is known to depend on at least six factors: total absorbed dose and pattern of delivery, radiosensitivity, rate of repair of sublethal damage, ongoing proliferation during treatment, tumor heterogeneity, and tumor size.

Depending on radionuclide characteristics, molecularly guided radiotherapy relies on radioactivity to destroy cells distant from immunotargeted cells. Therefore, even heterogeneous tumors (for antigen recognition) can be treated because not all cells have to be targeted. A single therapeutic strategy may be effective at some locations in the milieu of solid tumors but fail at other locations due to limitations in access of the drug, tissue hypoxia, or genetic heterogeneity. Accordingly, modifications to therapeutic protocol are to be expected by and within the expertise of one of ordinary skill in the art. All such modifications should be considered within the scope of the instant invention.

Anti-angiogenic therapies that target existing vasculature may be more effective, in some cases, than therapies that merely prevent new blood vessel formation. For example, a single existing blood vessel provides the nutrition for hundreds or thousands of tumor cells and the vessel need only be damaged at only one point to block blood flow to a majority of those tumor cells. Second, as the endothelial cells of a blood vessel are adjacent to the blood stream, there are little to no tissue barriers to drug delivery. Third, because the endothelial target of an existing blood vessel is presumably a “normal” cell, it is relatively unlikely to change its surface markers through genetic mutations. Finally, greater than 99% of tumor cells in vivo can be killed during a two hour period of ischemia.

However, anti-angiogenic therapy targetting both new and/or existing vessles can sometimes be rendered, in part, ineffective by regional heterogeneity, wherein tumor cells in the outer rim survive and re-grow, drawing nutrients from surrounding normal tissue vessels. In such cases, for an anti-vascular strategy to be ultimately successful, such stragegies preferably incude killing the outer rim of preangiogenic tumor cells. Thus, in one embodiment of the present invention, combination of antivascular attack and internal radiotherapy, achieved through combining short and longer range isotopes is contemplated. Indeed, a platform to localize multiple classes of isotopic radiation can be a powerful tool to control advanced cancer as it can address the heterogeneous cancer cell environment and allow individually tailored therapy.

Smaller tumors are more sensitive to molecularly guided radiotherapy than larger ones. A greater proportion of viable radiosensitive areas in small tumors, higher antibody uptake, and radiation dose, may each or all be responsible for this enhanced sensitivity to molecularly guided radiotherapy. In such an event, the effectiveness of therapy could be enhanced by matching the radionuclide with the delivery system target and tumor size.

Cancers that typically have p53 mutations (such as slow growing tumors generally, and breast cancer, in one specific example) are generally less susceptible to apoptosis, the apparent mechanism of cell death from low dose-rate radiation. In such cases, synergistic or combined modality radioimmunotherapy (CMRIT) agents may be used to increase radiosensitivity for therapeutic enhancement without additive toxicity. Antiangiogenic agents, which target normal, proliferating endothelial cells, have the potential to provide relatively nontoxic continuous inhibition of tumor growth by blocking new blood vessel growth and may synergize with molecularly guided radiotherapy to increase efficacy. Combined modality therapies (with αᵥβ₃ inhibitors, paclitaxel, docetaxel) may result in higher numbers of cures.

The construction, characterization, testing in-vivo of polymer conjugates, specifically APC, as well as endpoints for next stage of tumor therapy will now be discussed. The selected APC is specifically targeted to solid tumor neovasculature, has a low normal tissue residence, and is capable of carrying a flexible isotope payload including alpha, beta, gamma and positron emitting isotopes.

Synthesis and Characterization of Polymeric Conjugates

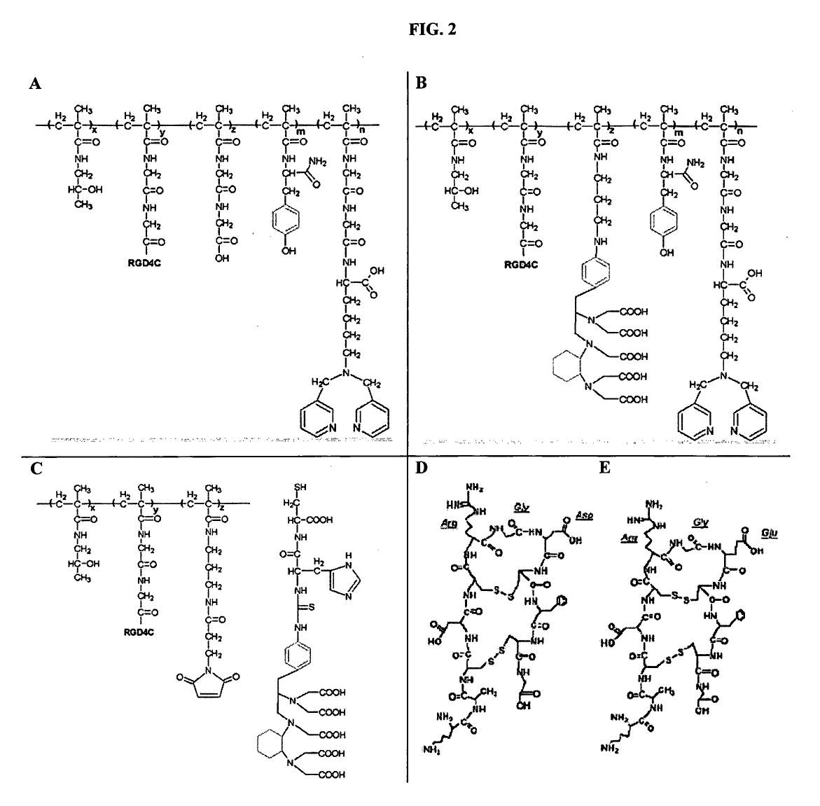

The doubly cyclized RGD peptide RGD4C, (FIG. 2) was prepared with a conformationally restrained RGD sequence that binds specifically and with high relative affinity to αᵥβ₃. The RGD binding site in the heterodimeric αᵥβ₃ integrin is located in a cleft between the two subunits. Hence, to reduce possible binding site steric hindrance from the HPMA polymer backbone, in one embodiment of the invention, a 26 atom glycylglycine (GG) chemical spacer between the polymer backbone and the RGD motif was introduced. However, it should be understood that chemical spacers may be varied in length and in composition depending on the steric considerations. As well, a chemical spacer may be used to space not only the targeting moiety, but also an isotope harboring moiety (collectively, “chemical moieties”).

To demonstrate tumor localization of HPMA copolymer conjugated RGD4C in mice models, several ^{99m}Tc labeled polymeric analogs including 1) polymer with targeting moiety (^{99m}Tc-HPMA-RGD4C), 2) polymer without targeting moiety (^{99m}Tc-HPMA) and 3) polymer with RGE4C, a control peptide analog with no affinity towards the αᵥβ₃ integrins (^{99m}Tc-HPMA-RGE4C) were constructed. The synthesis, characterization and radiolabeling of these and other polymer-peptide conjugates are described below.

The side chain components are shown in FIG. 2. Side-chain contents in the conjugates were consistent with their corresponding feed compositions during polymerization. Polymeric precursors containing reactive p-nitrophenyl ester (ONp) groups were first synthesized followed by coupling of the peptides (RGD4C/RGE4C) by aminolysis of the ONp groups with an average incorporation of 15 peptide moieties per polymer backbone. DPK derivatized peptides RGD4C-DPK (MW: 1583.1) and RGE4C-DPK (MW: 1598.5) were also synthesized and characterized to compare their biodistribution with the corresponding polymeric conjugates.

Polymer Backbone Labeling (Tc-99m)

One of the most stable technetium chelates in the form of ^{99m}Tc-tricarbonyl [^{99m}Tc(H2O)₃(CO)₃] was recently developed. This ^{99m}Tc-tricarbonyl core is thought to form relatively stable complexes with tridentate ligands by replacing the three coordinated water molecules. To permit stable radiolabeling of HPMA conjugates for scintigraphic imaging, a novel comonomer was developed, namely methacryloylglycylglycinedipyridyllysine (“MAGGDPK”) that was attached to the HPMA backbone.

HPMA copolymer conjugate ^{99m}Tc radiolabeling efficiencies were generally greater than about 93% with specific activities of about 16.8 to about 19.5 MBq/nmol. Similarly, radiolabeling of the DPK derivatized peptides yielded radiolabeling efficiencies of greater than about 95% with specific activities greater than about 8.3 MBq/nmol. The coupling of ^{99m}Tc-tricarbonyl complex to the DPK molecule provided a relatively compact tridentate coordination (FIG. 2) with no free coordination site for attack by competing ligands. Hence, such labeling was proposed to be stable in vivo. Indeed in vitro challenge studies of radiolabeled conjugates with competitive ligands such as cysteine and histidine indicated excellent ^{99m}Tc binding stability over 24 hours with less than 10% displacement (statistically insignificant) from the polymer backbone.

Endothelial Cell Adhesion Assay

The biological activity of the RGD4C peptide upon conjugation to HPMA was verified in vitro by an αᵥβ₃ positive Human Umbilical Vein Endothelial Cell (“HUVEC”) adhesion assay. This assay was developed on the understanding that mammalian adhesion proteins such as fibrinogen and vitronectin attach to cell surfaces through RGD motifs and this attachment could be competitively inhibited using RGD containing peptides. Briefly, HUVEC layers in 24-well plate were pre-incubated with binding buffer (50 nM Tris-HCl buffer supplemented with 150 nM NaCl, 1 mM CaCl₂, 1 mM MgCl₂, 1 mM MnCl₂ and 1% BSA). Subsequently, the cells were incubated with iodinated HPMA-RGD4C or HPMA-RGDfK and appropriate dilutions of free peptides for 4 hours at 4° C. The supernatant is sampled and cells washed, lysed and their radioactivity counted. Binding constants were calculated using non-linear regression to the Michaelis-Menten equation. IC₅₀ values may be calculated by non-linear regression.

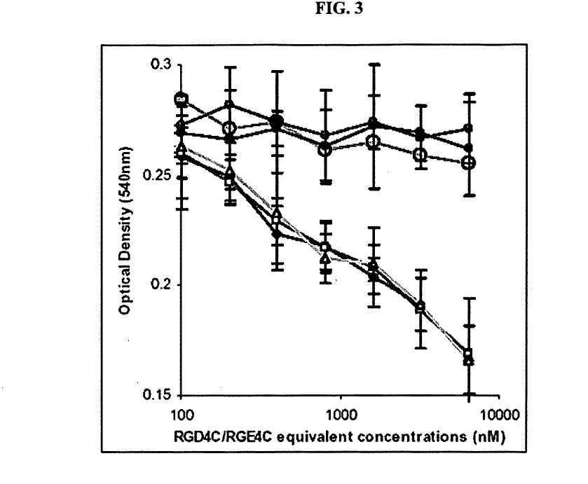

The results of the endothelial cell adhesion assays with the conjugates are summarized in FIG. 3. The results showed higher (p<0.05) inhibition of cell adhesion with HPMA-RGD4C conjugate compared to that of control HPMA-RGE4C, suggesting that bio-recognition may be mediated by RGD4C. At equivalent peptide concentrations, both free RGD4C and HPMA-RGD4C showed similar inhibition of adhesion, suggesting reduced loss in the peptide activity after conjugation to the polymer. The experiments also showed reduced loss of RGD4C bioactivity even after radiolabeling the conjugates with ^{99m}Tc. HPMA copolymer without any peptide attached, HPMA-RGE4C (the control peptide), and free RGE4C did not show marked inhibition of adhesion. These results further support the notion of highly specific RGD4C mediated binding to endothelial cells.

Evidence for In Vivo Targeting of APC Complexes

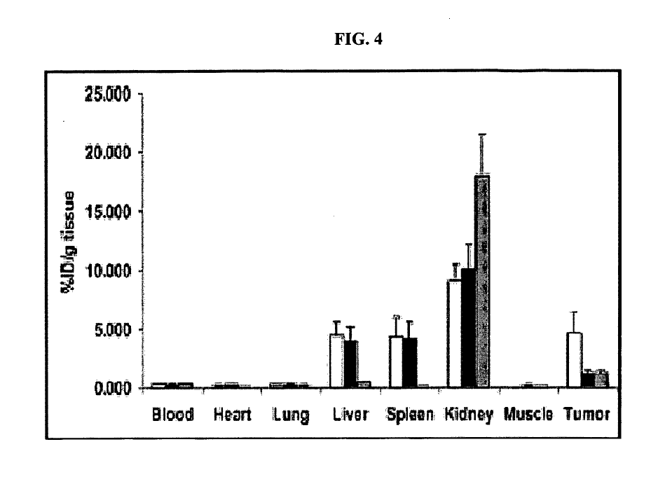

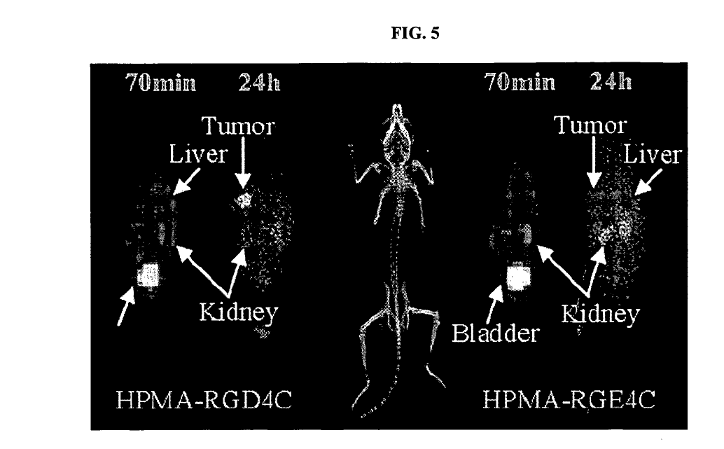

Specific nanohybrid targeting of tumor angiogenic vessels was evaluated in vivo by scintigraphic imaging and by tissue necropsy studies following administration of 99mTc labeled conjugates in SCID mice bearing human prostate tumor (DU145) xenografts. At 24 hours, the scintigraphic images (FIGS. 4-5) indicated significantly higher tumor localization of HPMA copolymer-RGD4C conjugate compared to HPMA-RGE4C conjugate. The quantitative biodistribution data of the ^{99m}Tc labeled copolymers 24 hours after administration showed the significantly higher (p<0.001) tumor accumulation of the HPMA-RGD4C conjugate (4.6±1.8% ID/g) than the HPMA-RGE4C conjugates (1.2±0.2% ID/g). Thus, for the first time, tumor vasculature targeting potential of HPMA-RGD4C conjugates was demonstrated as well as the use of dynamic scintigraphy to monitor the tumor localization and body distribution of these conjugates in real time.

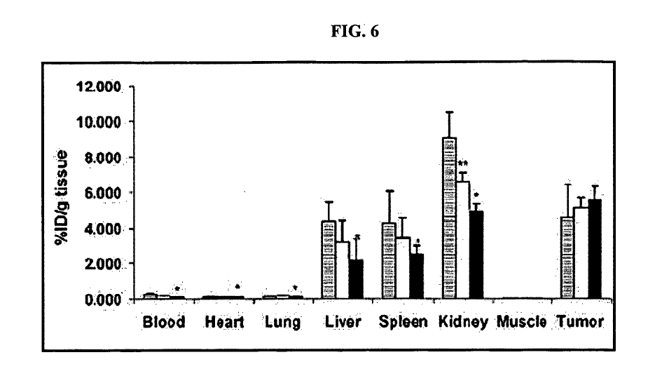

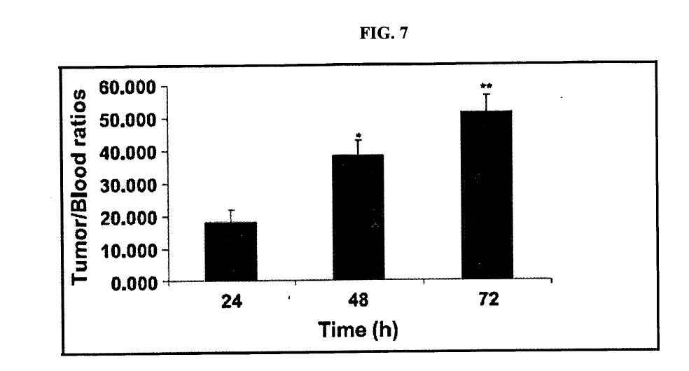

Long term biodistribution studies carried out over 72 hours (FIG. 6) showed a time dependent decrease of the polymer-RGD4C conjugate accumulation in liver, spleen and kidney. In contrast, a sustained tumor accumulation of the copolymer-RGD4C conjugate was observed over time. The tumor/organ ratios (FIG. 7) of the copolymer-RGD4C conjugate in the liver and spleen also increased significantly (p<0.05) with time. These data demonstrate the tumor targeting of RGD4C conjugates in vivo and the ability to image and evaluate the biodistribution of such conjugates.

Evidence for Improved Biodistribution of APC Complexes

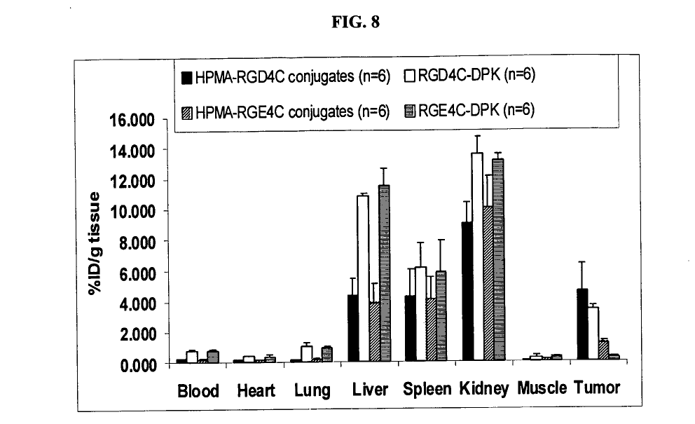

The biodistribution of HPMA-peptide conjugates with that of the free peptides (i.e. not attached to the polymer) were next compared. DPK derivatized free peptides were used to permit radiolabeling. Body distribution of prostate tumor bearing mice 24 hours post-injection of ^{99m}Tc-labeled conjugates are shown in FIG. 8. Enhanced tumor localization was seen for both HPMA-RGD4C and RGD4C-DPK compared to the controls (HPMA-RGE4C and RGE4C-DPK). However, the free peptides appeared to have background (liver, kidney) accumulation compared to the polymeric conjugates.

Predominant liver and kidney uptake of small RGD peptides as demonstrated by RGD4C-DPK is often considered a disadvantage of targeting and imaging tumor angiogenesis using small peptides. The biodistribution results suggest that conjugation of the RGD4C onto a polymer backbone (HPMA-RGD4C), however, can enhance the tumor/background tissue uptake ratio in comparison to the peptide itself, which can result in reduced systemic toxicity during therapy. This is likely attributable to 1) multivalency of the targeting moiety on the polymer backbone, 2) combination of active targeting and passive EPR effect of the macromolecular conjugate, and/or 3) decreased extravasation in normal tissues due to the large molecular weight of the conjugates. Low extravasation in normal tissues may result in reduced systemic toxicity during therapy. Hence, the enhanced tumor to background contrast of the polymer conjugate (HPMA-RGD4C) suggests that these constructs may have significant value as a means to target endothelia of tumor neovasculature.

Evidence for Effects of Polymer Charge and Size on Biodistribution

^{99m}Tc labeled electro-negative and neutral HPMA copolymers were synthesized and the effect of charge and molecular weight on their biodistribution in tumor-free SCID mice was characterized. Alternatively, biodistribution analyses may be conducted on electronegative conjugates in mice bearing model prostate tumor of high and low angiogenic response (e.g., PC3 and DU145). In any event, 7 kD, 21kD (below renal filtration threshold) and 70 kD (above renal threshold) HPMA copolymers containing a ^{99m}Tc chelating comonomer, bearing N-ω-bis(2-pyridylmethyl)-L-lysine (DPK), were synthesized by free-radical precipitation copolymerization for both the negative and neutral polymers.

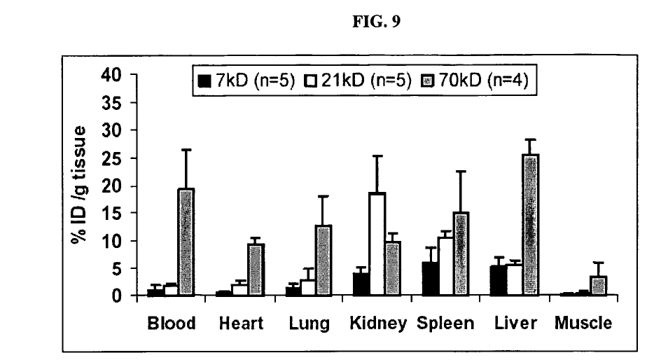

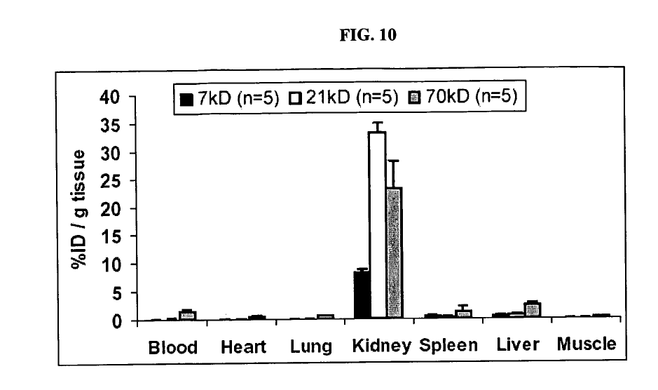

Necropsy data (FIGS. 9-10) showed that the negatively charged copolymer fractions were more efficiently cleared from the body than the neutral copolymers. In fact, the electronegative copolymers were not taken up substantially by any body organ other than the kidneys. All the neutral compounds showed hepatic activity, whereas little liver uptake was evident for the electronegative copolymer fractions. Without being limited to or bound by theory, these findings may be due, in part, to reduced transvascular flux from repulsion of the electronegative copolymers by negatively charged plasma membranes.

Similarly, the tissue retention of neutral HPMA appeared closely related to its copolymer molecular weight (FIGS. 9-10). For non-renal tissues studied (heart, lung, spleen, liver and muscle), the higher the molecular weight, the higher the tissue radioactivity. It is believed that increasing the molecular weight may improve tumor accumulation by EPR while maintaining satisfactory renal clearance to minimize non-tumor accumulation. Since EPR effect is known to be minimized by electronegative charge, this effect may be counterbalanced to some extent by increasing the molecular weight of the polymer.

Thus, the data demonstrated that stable chelation of ^{99m}Tc may be achieved by incorporation of a DPK-bearing comonomer into the HPMA backbone. Further, the data (FIGS. 9-10) showed that (1) negatively charged copolymers were eliminated from the body faster than neutral copolymers and (2) an increase in moleculare weight for neutral copolymers resulated in higher uptake by the cells of the reticuloendothelial system. Taken together, varying the size and charge of the polymeric carrier can alter biodistribution of the polymer-conjugate complex and thus accordingly tailored for optimal therapeutic effect. Preferable conjugates in some embodiments will have negative charge (most likely up to 40% COOH groups) and a molecular mass (most likely between 7-40 kD).

The above results may be used in designing a polymer backbone for targeted tumor delivery. In addition to the attachment of vascular endothelial cell specific targeting moiety, if necessary, it is possible to vary molecular weight and charge in order to alter biodistribution, minimize nonspecific uptake, and maximize localization in and around the tumor. Generally, to incorporate negative charge, polymer precursors may be synthesized with additional comonomer methacryloylglycylglycine (MAGGCOOH), which has a terminal negatively charged carboxyl group.

Alpha Emitters

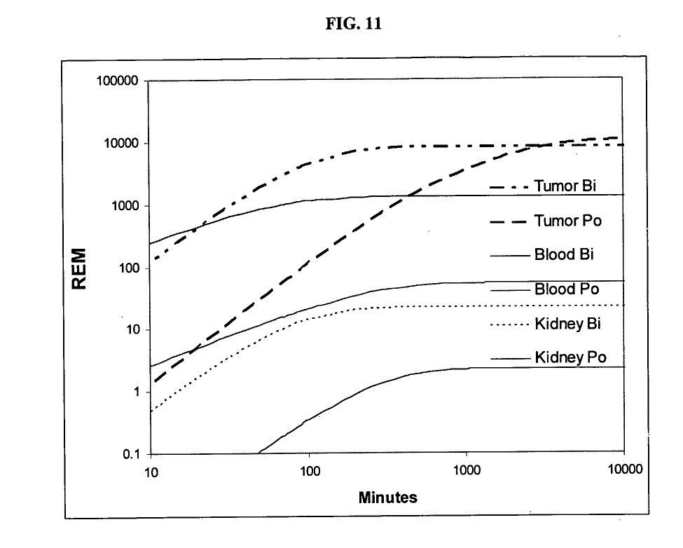

Based on our animal studies of APC biodistribution, the relative advantages of different alpha emitters (FIG. 11) were modeled. ²¹⁰Po was chosen in this embodiment because it emits a single alpha without other radiation and decays to stable ²⁰⁶Pb. Additionally ²¹⁰Po has uniquely clean emission, ease of production/availability and available purity (99.9%), chemistry, and the modeling predictions of its superiority over other shorter-lived alpha emitting isotopes. Generally, a preferable isotope is defined as one that can deliver the highest possible cytotoxic dose to the vessels and cells of cancerous tissue, while minimizing effects on surrounding non-pathological tissue. Relative to other sources of radiation, the use of angiogenesis targeted alpha-emitters is particularly advantageous for smaller tumors, disseminated disease, and metastatic disease, where specific localization is more critical to patient care.

### EXAMPLE

An HPMA copolymer was constructed with high electronegative charge to reduce liver and spleen localization. A molecular weight of about 40 kD was chosen to provide an intermediate circulation time to enhance targeting, yet retain a relatively rapid clearance to reduce non-tumor tissue residence time. The electronegative charge was introduced using the comonomer APMA-CHX-A″-DTPA. The final nanohybrid conjugate had a polydispersity of 1.42 and a MW=43.3 kD by SEC. The molecule contained 0.179 mmol/gm DPK, 0.077 mmol/gm Tyr, and 0.377 mmol/gm RGD4C by amino acid analysis. There was a mean of 16.3 RGD4C peptides per polymeric backbone.

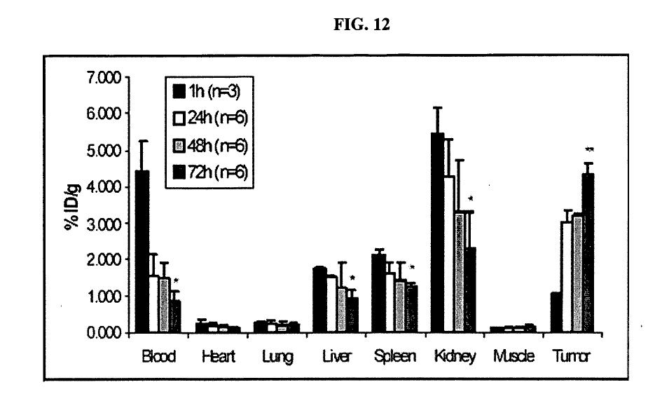

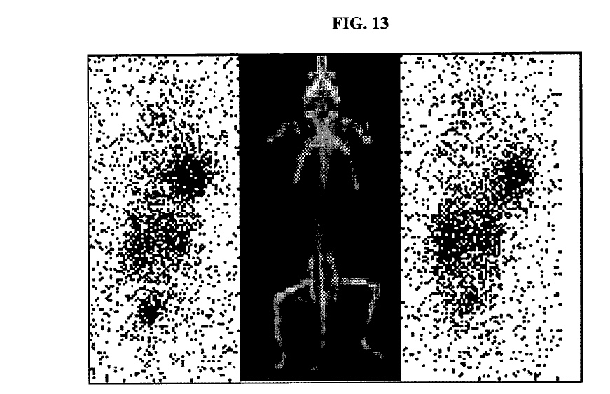

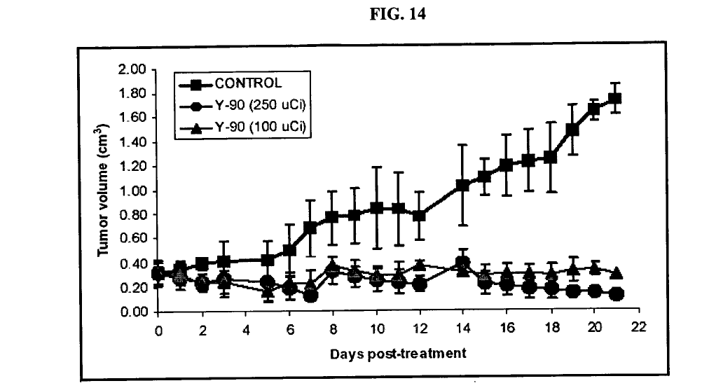

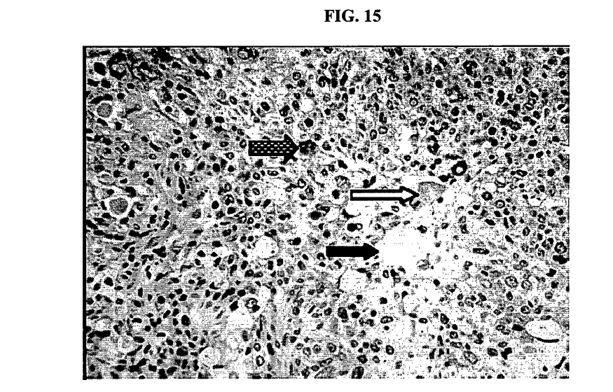

Yttrium-90 was chosen as the therapy isotope because of its attractive physical parameters (bmax=2.27 MeV, T_{1/2}=64 hr, X₉₀ [radius of sphere containing 90% of emitted energy]=5.34 mm) and because it binds very stably with CHX-DTPA. Three ⁹⁰Y treatment doses were chosen based on maximum tolerated dose levels defined in earlier studies of ⁹⁰Y radiolabeled antibody. 100 and 250 mCi ⁹⁰Y labeled polymer conjugates showed significantly higher (p<0.001) tumor growth inhibition as compared to the controls (FIGS. 12-15). At 21 days, the treatment groups showed 14.6 (250 μCi) and 5.8 (100 μCi) fold decrease in tumor volume as compared to the control. The mice in the treatment groups also showed higher (p<0.05) body weight loss than the controls. Histopathological examination of tumor sections post-treatment with 250 μCi ⁹⁰Y-copolymer conjugate showed greater cellular damage than the control. The tissues from other major organs, e.g., liver, kidney and spleen of the treatment animals were similar to the controls and showed no indication of toxicity.

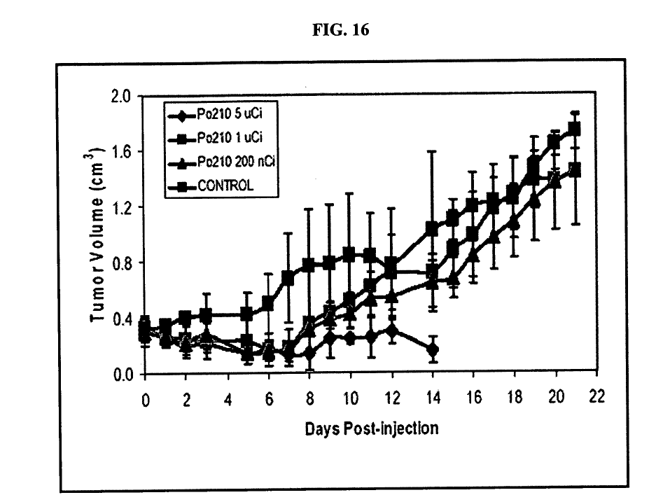

Therapy trial results (FIG. 16) showed similar tumor growth suppression during the first week following nanohybrid administration. Subsequently lower dose levels (1 and 0.2 μCi ²¹⁰ Po) showed a recovery of the growth pattern, presumably due to regrowth of the preangiogenic outer rim. These studies demonstrate the therapeutic relevance of the nanohybrid approach. Both a therapeutic entity (⁹⁰Y, ²¹⁰Po) and imaging agent (^{99m}Tc) were attached to the carriers to allow imaging and therapy. It is believed that such an actively targetable polymeric delivery system is the first of its kind to be developed.

Dose Calculation for Y-90 Microsphere Based on SPECT-CT Registration

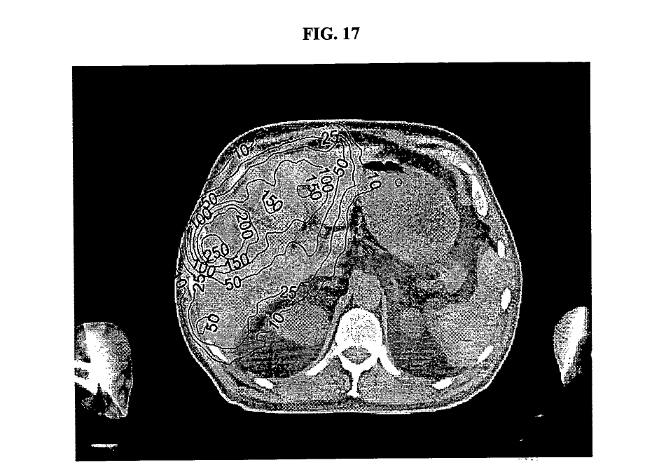

A technique was developed that uses SPECT and CT images to obtain 3-D dose distribution in patients with hepatic tumors treated with ⁹⁰Y microspheres infusion. The EGS4 Monte Carlo system with the PRESTA algorithm for electron transport may be used to calculate the dose spread kernels. With CT-SPECT fusion, the internal distribution of Y-90 revealed by SPECT is mapped three-dimensionally to CT. A convolution/superposition model is then used to calculate the dose distribution, which provides complete description of the radiation-absorbed dose to the liver and tumor. FIG. 17 shows the CT scans of a patient treated with ⁹⁰Y doped microsphere for hepatocellular carcinoma.

The isodose distribution superimposed on CT scans may serve as a tool to plan a patient's treatment, guide the treatment procedure, and evaluate the treatments prognosis and efficacy. For example, a conventional prescription assumes that activity to be distributed uniformly within the liver. However, a calculated dose-volume histogram indicates that only 13% of the normal liver (excluding the tumor) received a dose higher than 114 Gy. In comparison, 62% of the tumor received more than 114 Gy. Based on the dose distributions, dose analysis tools, such as dose-volume histograms, conventionally used in external beam radiation therapy can also be used for internal irradiation.

In some embodiments, preferable conjugates may be optimized for i) highest tumor accumulation (>4% injected dose within 6 h) and ii) best normal tissue clearance (Tumor/Background ratio of >10). It is known that water-soluble polymeric carrier like HPMA prolong the circulation life of a drug/radiotherapeutic agent and increase accumulation in the angiogenic tissue through the EPR mechanism. However, it has been demonstrated that such a molecule may also need to be relatively electronegative (equivalent of approximately 8 mole % COOH groups) in order to be effectively cleared from circulation and thereby minimize non-tumor tissues/organs. In addition, rapid clearance is contingent upon the hydrodynamic radius (less than 45 Å) and the molecular weight of the polymers (less than 45 kD) being below the threshold of renal filtration.

In other embodiments, polymer conjugates include HPMA copolymer-(DPK)(HBA)(DOTA)(RGD4C) conjugates. The content of the targeting peptide (RGD4C) is preferably kept constant at 20 mole % (˜15 units of the peptide per HPMA chain) as this provides multivalency and effective tumor localization. The polymer backbone may constitute a variety of chelators capable of incorporating one or more radioisotopes. These may include a) dipyridyllysine (“DPK”) for chelating ^{99m}Tc (the imaging agent that may allow monitoring the biodistribution of these conjugates in vivo by gamma scintigraphy), b) m-hydroxybenzoic acid (“HBA”) for chelating Iodine isotopes (¹³¹I and ¹²⁴I) and c) 1,4,7,10-tetraazacyclododecane-1,4,7,10-tetraacetic acid (“DOTA”) for chelating the therapeutic alpha emitter ²¹⁰Po and therapeutic beta emitter ⁹⁰Y. These are established chelators for the respective isotopes. DPK, and HBA can be incorporated in small molar proportions of 5% each to enable satisfactory labeling. The DOTA molecule contains four carboxyl (“COOH”) groups resulting in its overall electronegative character. Hence the DOTA content of the polymer constructs is preferably incrementally varied at about 5 to about 7.5 to about 10 mole % to obtain the desired electronegative charge for clearance. This introduces a charge equivalent of 20, 30 and 40 mole % COOH groups, respectively.

In other embodiments, preferable conjugates will meet both the following criteria:
A statistically significant tumor accumulation (greater than about 4% injected dose/g), demonstrated within 6 hours.
A relatively high tumor to normal tissue (T/N) uptake ratio (greater than about about 10) after 24 hours. The desired liver and kidney uptake should be less than about 1%. In some embodiments, the polymer composition with the least negative charge is desirable.

Preferable polymers may be defined in terms of its content of RGD4C (expressed as millimoles/g of polymer) and electronegativitiy (expressed as millimoles DOTA/g of polymer) as well as the dose in peptide equivalents required for significant tumor localization. The lead conjugate may then be radiolabeled with the different therapeutic isotopes, e.g., alpha (²¹⁰Po), or beta (⁹⁰Y, ¹³¹I). In some embodiments, isotope conjugate(s) alone or in combination that demonstrate the highest antitumor efficacy are desirable.

Synthesis and Characterization of the Comonomers

HPMA is a monomer that may, in one embodiment, render the polymer water-soluble and constitute a significant portion of the polymer backbone. Methacryloylglycylglycyl-paranitrophenyl ester (MAGGONp) is a reactive comonomer to which vascular targeting peptide RGD4C may be attached after polymerization. Methacryloylglycylglycyldipyridyllysine (MAGGDPK) is a comonomer that chelates ^{99m}Tc for in vivo scintigraphic imaging and biodistribution studies and ¹⁸⁸Re (a therapeutic beta emitter). N-methacryloyltyrosinamide (MA-Tyr) is a comonomer which may be used to chelate Iodine isotopes.

Methacryloylglycylglycyl-p-aminobenzyl-1,4,7,10-tetraazacyclododecane-1,4,7,10-tetraacetic acid (MAGGDOTA), a comonomer that renders electronegative charge to polymers and also chelates ⁹⁰Y (therapeutic beta emitter) and ²¹⁰Po (therapeutic alpha emitter): To a solution of p-aminobenzylDOTA in dry dimethyl sulfoxide (DMSO), a stirred solution of MAGGONp in DMSO is added at a 2:1 molar ratio in the presence of t-octyl pyrocatechine inhibitor. The reaction mixture is continuously stirred at room temperature for 24 hours. The DMSO is roto-evaporated; the crude comonomer purified by washing with ether and recrystallized from methanol. The comonomer is then dried under vacuum and characterized using UV spectrometry (Imax=238 nm, e=8737 M⁻¹cm⁻¹) and mass spectrometry (MW 691).

Methacryloylglycylglycylethylhydroxybenzoate (MAGGHBA), a comonomer to chelate iodine isotopes with reduced possibility of dehalogenation: m-hydroxybenzoic acid (HBA) is derivatized with N-hydroxysuccinimide (NHS) in presence of dicyclohexylcarbodiimide (DCC) to get NHS ester of HBA (NHS-HBA). The comonomer containing HBA is synthesized as follows: MAGGONp is derivatized with ethylenediamine by aminolysis of ONp; NHS-HBA is then reacted with MAGGethylenediamine to get MAGGHBA.

N-methacryloylaminopropyl-2-amino-3-(isothiourea-phenyl)propyl-cyclohexane-1,2-diamine-N,N—N′,N′,N″,N″-pentaacetic acid (APMA-CHX-A″-DTPA) is synthesized by reacting p-SCN-CHX-A″-DTPA in dry dimethyl sulfoxide (DMSO) with N-(3-Aminopropyl)methacrylamide hydrochloride (APMA), in presence of N,N-diisopropylethylamine at room temperature for 24 hours under nitrogen. The pure product can be be isolated by silica gel column chromatography (Silica Gel 60), eluted with 2-propanol:water:NH₄OH (8:1:1) followed by 2-propanol:water:NH₄OH (7:2:1). The solvent is then vacuum evaporated, excess ether added to precipitate the product, and the precipitate filtered and dried under vacuum. The product was characterized by mass spectrometry (MW 736.72), UV spectrometry (λmax=274 nm, ε=8811.58 M⁻¹cm⁻¹ in DMSO), and TLC (Rf=0.61, 7:2:1 2-propanol: water: NH₄OH).

Iodine coupling comonomer, N-methacryloylaminopropyl-3-hydroxy-benzoate (APMA-HBA) may be synthesized in two steps. First, 3-hydroxy-benzoic acid is derivatized to N-succinimidyl 3-hydroxy benzoate using dicyclohexylcarbodiimide as a coupling agent in dry DMF. Second, N-succinimidyl 3-hydroxy benzoate is reacted with APMA in presence of N,N-diisopropylethylamine in dry DMF for 24 hours at 22° C. The pure product is then isolated by silica gel column chromatography (Silica Gel 60, E. Merck, Germany). Finally, the solvent is vacuum evaporated, excess ether added to precipitate the product, and the precipitate filtered off and dried under vacuum.

HPMA copolymers bearing CHX-A″-DTPA or DOTA may be synthesized by free radical precipitation copolymerization of comonomers in dimethyl sulfoxide (DMSO) in acetone using N,N′-azobisisobutyronitrile (AIBN) as the initiator. The feed composition of the comonomers is be kept at about 95 mol % for HPMA and about 5 mol % for APMA-CHX-A″-DTPA or APMA-DOTA. The comonomer mixtures are sealed in an ampoule under nitrogen and stirred at 50° C. for 24 hours. The precipitated copolymeric precursor is then dissolved in methanol and re-precipitated in acetone:ether (3:1) to obtain the pure product.

The copolymer may be characterized by weight average molecular weight (MW) and molecular weight distribution (polydispersity, calculated as weight average Mol. Wt./number average Mol. Wt.) by size exclusion chromatography (SEC) on a Superose 12 column (10 mm×30 cm) using a fast protein liquid chromatography (FPLC) system (Amersham Biosciences). HPMA copolymers containing 3-hydroxybenzoic acid (HBA) may be synthesized by polymerizing 98 mol % HPMA and 2 mol % APMA-HBA as described above. The content of CHX-A″-DTPA, DOTA and HBA may be calculated by UV spectrometry.

Synthesis and Characterization of the Polymeric Precursors

Polymeric precursors containing MAGGDPK, MA-Tyr, MAGGHBA, MAGGONp, APMA-CHX-A″-DTPA and varying molar amounts of MAGGDOTA may be synthesized by radical precipitation copolymerization of the comonomers in appropriate molar feed ratios. The molar feed amount of MAGGONp preferably corresponds to the desired RGD4C content in the final conjugate. N,N′-azobisisobutyronitrile (AIBN) can be used as a free radical initiator. Briefly, comonomers are dissolved in acetone/DMSO and transferred to an ampule, bubbled with nitrogen for 5 min. and sealed. Polymerization is carried at 50° C. with stirring for 24 hours. The polymer is then dissolved in methanol and reprecipitated (2×) in ether.

The weight average molecular weight (Mw) and molecular weight distribution (polydispersity) of the precursors may be estimated by size exclusion chromatography using, for example, a Superose 12 column (10 mm×30 cm) with a fast protein liquid chromatography (FPLC) system.

To synthesize polymers with varying molecular weight, the crude polymer above is fractionated by elution on a Superose 12 preparative column (16 mm×50 cm), using PBS (pH 7.4). The fractions are dialyzed against distilled water and lyophilized. The overall electronegative charge on the polymers are varied by varying the feed ratio of MAGGDOTA. The content of reactive ONp functional group of polymeric precursors can be determined by UV spectrophotometry (e272 nm=9500 M⁻¹cm⁻¹). The content of DPK, Tyr, DOTA is determined from the amino acid analysis.

Synthesis and Characterization of Polymer-RGD Conjugates

The vascular endothelial bed targeting peptide (RGD4C) may be attached to the polymeric precursors by aminolysis of the terminal ONp groups of the MAGGONp side chains of the corresponding precursors. Briefly, to a solution of polymeric precursors of HPMA in dimethylformamide (DMF), a solution of RGD4C in DMF is added. After stirring overnight (16 h) in the dark at room temperature, a slight excess of aminopropanol is added and stirred for an additional hour to neutralize any unreacted ONp groups. The crude conjugates are dissolved in methanol and precipitated in ether. The precipitation should be repeated twice to remove unreacted drug and peptide. The crude polymer is dissolved in deionized water, dialyzed and finally lyophilized. The content of RGD4C may be determined by amino acid analysis and expressed as millimoles of peptide per gram of polymer conjugate.

HPMA copolymer-peptide conjugates may also be synthesized in a two step procedure as follows: First, HPMA copolymer precursor containing 20 mol % MAGG-ONp, 5 mol % APMA-CHX-A″-DTPA or APMA-DOTA, 2 mol % APMA-HBA and 73 mol % HPMA is synthesized as described above. The contents of APMA-HBA is determined by UV spectrometry. APMA-CHX-A″-DTPA or APMA-DOTA content is determined by acid-base titration. MAGG-ONp content is assessed by release of ONp from the polymer in 1.0 N sodium hydroxide by UV spectrophotometry (400 nm). Second, HPMA copolymer precursor is conjugated to either RGD4C or RGDFK via p-nitrophenyl ester aminolysis of the polymeric precursor.

Briefly, polymeric precursor in dry DMF and dry pyridine is added under constant stirring to RGD4C or RGDfK and continuously stirred at room temperature for 22 hours. The reaction is terminated with 1-amino-2-propanol. The crude conjugate is dialyzed against deionized water and lyophilized. The peptide content in the conjugate may be analyzed by amino acid analysis and the conjugate molecular weight may be determined by SEC.

Determination of Binding Efficacy of Targetable Polymer Conjugates

The biological activity (“biorecognition”) of the targetable polymer-RGD4C conjugate may be assessed in vitro using a modification of standard cell adhesion assay. The binding of Human Umbilical Vein Endothelial Cells (HUVEC) to fibrinogen is mediated by αᵥβ₃ and therefore can be competitively inhibited by RGD4C. This property may be exploited to analyze the relative binding of RGD4C containing polymer conjugates. The methods have been described in detail elsewhere.

Radiolabeling of Polymer-Peptide Conjugates with Alpha, Beta, Gamma and Positron Emitting Radioisotopes

Polymer-peptide conjugate may be radiolabeled with different imaging and therapeutic isotopes to demonstrate the feasibility of labeling and stability under physiological conditions. The radiolabeling efficiency is calculated as: (activity of labeled polymer)/(activity added)×100. The specific activity is calculated as activity labeled on the polymer/peptide per mg weight.

Both HPMA-CHX-A″-DTPA and HPMA-DOTA copolymers can be labeled with ⁹⁰Y or ¹¹¹In. Alternatively, these copolymers may be labeled with ²¹⁰Po by incubating polymer solution in acetate buffer (pH 5.0) with ²¹⁰Po at room temperature, 45° C. and 100° C. for 30 and 60 min. The labeled polymers may be purified using Sephadex G-25 column (PD-10 desalting column, Amersham Biosciences, Piscataway, N.J.). The labeling stability may be evaluated using in vitro by incubating the labeled polymers in human serum at 37° C. HPMA-HBA copolymer may be labeled with ¹²³I, ¹²⁴I and ¹³¹I using the Iodogen method.

Radiolabeling and Stability Studies with ^{99m}Tc

These isotopes are chelated to the DPK molecule. General procedures are known in the art. The stability of chelation may be estimated by cysteine and histidine challenge studies.

Radiolabeling and Stability Studies with ⁹⁰Y and ²¹⁰Po

These isotopes are chelated to the DOTA molecule. By one of one example, 1-5 mCi of radioisotope in 25 ml of 0.05M HCl are buffered with 128 ml of labeling buffer (0.2M ammonium acetate, pH 5.0). 1 mg of polymer conjugate 40 ml acetate buffer is then added. In some embodiments, 0.1 and 0.01 mg of HPMA conjugate may be desirable to increase specific activity. The resulting mixture is incubated at 100° C. for 30 min. Equal volume of 10 mm DTPA is then added and incubated at room temperature for an additional 15 min. to chelate any unbound radioisotope. The labeled conjugate is purified by size-exclusion chromatography using a PD-10 desalting column. The samples may be counted on a scintillation counter using appropriate cocktail (Instagel for Po, Scintisafe 30% for Y). The stability of the radiolabeled conjugates can be estimated by DTPA challenge studies.

Radiolabeling and Stability Studies with ¹³¹I, ¹²⁴I

The HBA containing side chains on the polymer can be radioiodinated as known to one of ordinary skill in the art using IODOGEN beads.

Scintigraphic Imaging and Biodistribution Studies

In one embodiment, four to five week old male Harlan Sprague-Dawley SCID mice (average weight 25 g) are anesthetized and injected via the lateral tail vein with about 200 μl of normal saline containing 25 nmol of various ^{99m}Tc-HPMA conjugates (300-400 μCi). To assess the early organ biodistribution, a dynamic 90 min image may be obtained immediately after intravenous injection using a dual head gamma camera with a low energy all-purpose collimator (DSX-LI SMV). Also, at 6 hours and 24 hours, 30-min static scintigraphic images may be obtained to evaluate residual organ activity. At 24 hours, animals will be necropsied, and whole organ tissue samples will be obtained from the heart, lung, liver, spleen, kidney, muscle and tumor. The tissue samples will be washed with water, counted (Cobra II Autogamma), weighed, and the %-injected dose per gram tissue (% ID/g) is calculated. The biodistribution studies may be performed additionally at 48 hours and 72 hours to demonstrate the kinetics of distribution by drawing time activity curves.

Without being limited to or bound by theory, an increase in the content of RGD4C in the polymer side chains may result in higher biorecognition up to a saturation point; inefficient binding could result if the peptide content is low. Moreover depending on the receptor density of the αᵥβ₃ integrins, increasing peptide content is likely to induce significantly enhanced binding at low concentrations due to multivalency. If suboptimum binding is observed, the corresponding polymeric conjugates may need to be resynthesized with increased peptide content.

In one embodiment of the instant invention, tumor models of diverse morphologies and growth characteristics are used with autoradiographic analysis to evaluate the relationship between APC tissue localization, APC radioisotope payload, APC effective radiation dose and histopathological effect in both tumor and normal tissues. Such tumor models include, for example, PC-3, DU-145, LnCaP, and LuCaP23.2 (hormone dependent). Histopathology may include assessment of gross morphological effects, endothelial injury/vascular thrombosis, and presence of morphological and molecular signs of apoptosis.

The therapeutic value of the APC is assessed in some embodiments by evaluating the therapeutic polymer (APCRx) tumor/normal tissue microdistribution when armed with a and/or b emitting radioisotopes. Specifically, the therapeutic impact of APCRx is determined by correlating α and/or β radioisotope dosage with autoradiographic and histopathologic analyses.

In some embodiments, it may be desirable to identify the maximum tolerated dose (MTD) of APCRx conjugates containing the respective radioisotopes. A range of incremental doses of each isotope is available. The MTD for each isotope is defined as the dose at which 100% of the animals survive with less than 20% weight loss, and that at the next higher dose level, at which >10% animals either died or experienced >20% loss in body weight.

Histopathological evaluations may be done at the MTD. The studies are done in mice bearing tumors of different morphology and histology namely micrometastatic tumor and solid tumor of both high and low angiogenic response. Animals are observed until death or a loss of greater than 30% of their original weight. The period of study need not exceed 2 months. During the first week of treatment one animal is on Day 3 and Day 7 and necropsied for alpha or beta dosimetry. Histopathological evaluations are done on tissue specimens following necropsy. Histological evaluations are similarly done upon death of mice during the entire period of study. The histological data for each isotope bearing polymeric conjugate is correlated to the corresponding first week microdosimetry data.

The information gained from the alpha and beta phases may be used to define a strategy for combined alpha and beta source radiotherapy. In some embodiments, <about 42% of control, and whose “control” polymers not bearing RGD targeting sequences do not show activity are desirable.

Determination of MTD

Normal non-tumor bearing SCID mice are injected with incremental dose levels of the Rx isotopes and survival and mice weight are monitored daily for up to 4 weeks post treatment and compared with a control group.

Animal study for evaluation of tumor dosimetry and histopathological changes. Once MTD has been determined for each agent microdosimetry, histopathological evaluations may be done at the MTD. These studies may be done in mice bearing both micrometastatic tumor and solid tumor of high and low angiogenic response. Typically tumor bearing nude mice in groups of 15-20 may be given single i.v. injection per dose and monitored over two months. To determine the effect on tumor and tumor size reduction a control untreated group of tumor bearing animals may be used for comparison. Tumor volume may be monitored weekly. If tumor exceeded 5 cm³ the animal will be removed from the study. Animals should also be removed if the overlying skin or tumor became ulcerated. All animals otherwise should be monitored till death occurs.

For microdosimetric analysis one animal can be sacrificed each, for example, on Day 3 and Day 7 and necropsied. Several physiological and biochemical parameters may be monitored on a weekly basis including, hematology, BUN, creatinine, glutamate oxaloacetate transaminase and alkaline phosphatase levels assayed as described previously. Histopathological evaluations may be done as known in the art following death of animals, and tumor dosimetry may be performed using autoradiographs of the excised tumors.

Autoradiography

Autoradiography may provide the regional distribution of the radionuclides in tumors. A Monte Carlo calculated point dose function may be generated for each alpha and beta emitter used in this study. The radiation absorbed dose rate distribution within the tumor may be calculated by convolving the activity distribution with radionuclides point dose function. Autoradiography only provides a snap shot of the activity distribution. A temporal distribution of the activity to calculate the accumulated dose in tumor may be desirable. Serial whole body scintigraphy may be used to obtain the activity-time dependency. Whole body, undecalcified sections of the animals may be performed in order to have better mapping correlations between dosimetry and histopathology.

Tumor counts may be fit into a multi-exponential function and a resident half life for the compound may be derived. Micro-TLD may be implanted into the tumor so to record accumulated dose at implanted point. The absorbed dose calculated with autoradiography can be scaled to the TLD point doses to obtain total dose to tumor cells. The resultant dose distribution superimposed on histological images may provide dose-tumor damage relationship at micro scale. In addition to dose calculations for tumors, the dose to critical organs (kidney, liver and bone marrow) that might limit tumor dose may also be calculated.

Tumor Histopathology

Tumor treatment injury effect may be analyzed with emphasis on: (a) the relationship of the tumor cells to the vascular bed, distinguishing between the central area of the tumor with prominent hypoxic conditions and lack of angiogenesis; the outer shell of the tumor, receiving coverage from both neoangiogenesis as well as surrounding milieu and the intermediate zone between the tumor center and the outer shell, characterized by prominent neoangiogenesis; (b) the degree of tumor cell and endothelium injury including a spectrum of changes ranging from milder injury characterized by thanatosome (hyaline cytoplasmic globule) formation to biochemical expression of activation of the apoptosis program (caspase activation) to the overt phenotype of cell apoptosis by H&E morphology and demonstration of apoptotic DNA cleavage; (c) the proliferative potential of tumor cells.

It is expected that mild injury in well perfused tumor areas may produce mostly cytoplasmic hyaline globule (thanatosome) formation. This form of injury involves autophagocytosis of mitochondria and other organelles as well as plasma membrane changes. The thanatosomes are histochemically intensely perjodic acid Schiff (PAS) positive, thus better suitable for image analysis. The opposite end of this spectrum may show in avascular tumor areas confluent hypoxic cell necrosis. Marked treatment effect in well perfused areas may show prominent morphological and biochemical features of overt apoptosis in addition to thanatosome formation.

Specifically, the area of gross tumor necrosis may be measured and calculated as percentage of the total tumor surface. The tumors may be sampled for histological examination from the following areas: (a) central (b) outer shell (c) intermediate zone between (a) and (b). In each of these areas the following parameters may be assessed by simple microscopic examination and computerized image analysis: 1) Percent of confluent tumor cell necrosis (by H&E morphology); 2) Apoptotic Index (AI): Number of tumor cell apoptosis/high power field (by H&E morphology) (20 high power fields examined of tumor areas without confluent necrosis); 3) Tumor cell mitotic index (MI) (number of mitoses/high power field (hpf)); (4) Degree of thanatosome (cytoplasmic hyaline globule) formation semiquantitatively (assessed as: 0 (no TS/10 hpf), 1+(1-2 TS/10 hpf), 2+(3-5 TS/10 hpf) and 3+(>5 TS/10 hpf)); 5) Vascular fibrinoid necrosis and/or thrombosis semiquantitatively assessed as: 0 (no necrosis and/or thrombosis), 1+(1-2 necrotic and/or thrombotic vessels/10 hpf), 2+(3-5 necrotic and/or thrombotic vessels/10 hpf), 3+(>5 necrotic and/or thrombotic vessels/10 hpf); 6) Tumor cell growth fraction (cycling population) by immunohistochemical detection of the Ki-67 antibody in tumor cell nuclei by immunoperoxidase/1,000 tumor cells (MIB-1 Labeling index); 7) Analysis of apoptosis by the TUNEL method (TUNEL index), wherein positive nuclei may be counted/hpf and separated as endothelial cells or tumor cell nuclei; 8) Analysis of apoptosis may be performed by immunohistochemical analysis of cleaved Caspase-3 (Caspase-3 index). Positive cells may be counted/hpf and separated as endothelial or tumor cells. 9) The following composite indexes may be also calculated: AI/MI, TUNEL/MIB-1 in order to assess the tumor cell population dynamics.

A cumulative treatment effect scoring system may be used as follows: All parameters may be converted for that purpose to a 0-3+ scale: (a) For tumor cell injury an aggregate score composed of the elements of Confluent tumor cell necrosis, AI/MI, Thanatosome formation, Tunel/MIB-1, and Cleaved caspase-3 index (0-15). (b) For vascular injury a similar score may be calculated composed of: Vascular fibrinoid necrosis/thrombosis, endothelial cell TUNEL and cleaved caspase-3 positivity (0-9).

Normal Tissue Histopathology

Sections of kidney, liver, lung and spleen may be analyzed for potential toxicity as follows: Kidney: a. Presence or absence and severity of acute tubular necrosis (0-3+). b. Presence or absence and severity of glomerular thrombosis or fibrinoid necrosis (% of glomeruli). c. Analysis of vascular fibrinoid necrosis and/or thrombosis, as previously described for the tumors. A piece of the kidney cortical parenchyma may be saved for potential electron microscopy studies. Liver: a. Presence or absence of necrosis (zones 1, 2 or 3 or confluent necrosis) quantified as percentage of liver tissue surface involved. b. Presence or absence of veno-occlusive disease (% of vessels involved). c. Liver parenchymal cellular injury expressed as: ballooning steatosis, apoptosis (Councilman bodies), Mallory bodies, thanatosomes (cytoplasmic hyaline globules), induction cells (% of parenchymal cells involved/zone). d. Analysis of vascular fibrinoid necrosis and/or thrombosis, as previously described for the tumor. Lung: a. Presence or absence and degree of diffuse alveolar damage. b. Analysis of vascular fibrinoid necrosis/thrombosis, as previously described for the tumors. Spleen: a. Presence of confluent necrosis (% of tissue involved). b. Analysis of vascular fibrinoid necrosis/thrombosis, as previously described for the tumors. Heart intestine & brain: a. Presence of tissue necrosis (% of tissue involved). b. Analysis of vascular fibrinoid necrosis/thrombosis, as previously described for the tumors.

Assessment of Acute Organ Toxicity

The following cumulative scores (each of the added scores converted to 0-3+) may be calculated: Kidney: Acute tubular necrosis (ATN), glomerular thrombosis/fibrinoid necrosis, and extraglomerular vascular fibrinoid necrosis/thrombosis (0-9). Liver: Zonal necrosis, venoocclusive disease, hepatocellular injury, and vascular fibrinoid necrosis/thrombosis (0-12). Lung: Diffuse alveolar damage (DAD), and vascular fibrinoid necrosis/thrombosis (0-6). Spleen: Confluent necrosis, and vascular fibrinoid necrosis/thrombosis (0-6). Heart, intestine & brain: Tissue necrosis, and vascular fibrinoid necrosis/thrombosis (0-6 in each organ).

Assessment of Chronic Organ Toxicity

The following cumulative scores may be collected in all organs above: Degree of interstitial fibrosis (gliosis for the brain), degree of parenchymal loss, vascular sclerosis (0-9).

For assessing renal and liver function, 200 μl blood may be collected in non-heparanized vials, and centrifuged for 10 min to collect the serum supernatant. Blood urea nitrogen may be determined by urease/glutamate dehydrogenase assay, glutamate oxaloacetate transaminase activity by combined asparatase aminotransfase/malate dehydrogenase assay and alkaline phosphatase activity using paranitrophenyl phosphoric acid as substrate.

To assess blood counts, blood samples can be collected in heparanized vials and diluted 1:200 in PBS (containing 0.9% saline/10 mM sodium phosphate) for RBC count; 1:100 in 1% ammonium oxalate for platelet counts and 1:20 in 3% acetic acid for WBC counts. To assess the in vivo biodistribution of beta and alpha emitters, 50 mg of tissue may be incubated with 0.5 ml Solvable (Packard Bioscience Company, Meriden, CT) at 50° C. for 3 hours. Thereafter, 0.1 ml of 30% hydrogen peroxide is added and incubated for another hour at 50° C. The samples are allowed to cool and 10 ml of scintillation cocktail is added and counted in a scintillation counter.

Necropsy Tissue Preparation

Tissue (heart, lung, liver, spleen, intestine, and kidneys) may be fixed in 10% buffered formaldehyde (pH 7.4), dehydrated in graded series of ethanol, immersed in petroleum, and embedded with random orientation in paraffin wax at a temperature of between 60° C. and 70° C. The paraffin-embedded tissue blocks were sectioned at a thickness of 4-5 mm and stained by H&E (hematoxylin and eosin) and evaluated histopathologically for physiological changes.

Tissue (heart, lung, liver, spleen, intestine, injection site, and kidneys) may be fixed in 10% formalin and evaluated histopathologically. In the event that any animal does not survive the full 2 month period, tissue (heart, lung, liver, spleen, intestine, and kidneys) may be fixed in 10% buffered formaldehyde (pH 7.4), dehydrated in graded series of ethanol, immersed in petroleum, and embedded with random orientation in paraffin wax at a temperature of between 60° C. and 70° C. The paraffin-embedded tissue blocks may be sectioned at a thickness of 4-5 mm and stained by H&E (hematoxylin and eosin) and evaluated histopathologically for physiological changes.

Model of Micrometastasis

Because cells injected i.v. via the tail vein must pass through the mouse lung before entering the general circulation, the mouse lungs may be examined FTUNEL for signs of colonization. Other organs including liver, spleen, kidney may also be examined for colonization. Studies on other tumor cell lines have clearly demonstrated that selective colonization of specific target organs is not simply the result of nonspecific trapping of tumor cells by the organ vasculature. One of the best studied examples is the B16 melanoma cell line, in which sublines have been isolated that preferentially metastasize to the lung and liver.

As an alternative to a vascular metastatic model, LNCaP tumor cells may be mixed with Matrigel (Becton Dickinson Labware) and xenografted into athymic nude mice, 8 weeks of age following the procedure described by McDevitt. In this technique mice receive an i.m. injection of 6-7E6 LNCaP tumor cells mixed with Matrigel in the right hind leg at a volume of 0.25 ml. Tumor growth in vivo may be assessed histologically at days 2, 3, 5, 7, and 10. At the time APCRx is administered (day 2), the tumors are disorganized cell clusters and nodules each comprised of several thousands of cells. The nodules are not vascularized and not encapsulated. On day 3, the tumors are more organized and are becoming vascularized, but still not encapsulated. By the 5th day, vascularization is more pronounced, and on day 7 the tumors are encapsulated.

To characterize this model, tissue sections may be stained by immunohistochemistry to detect multiple micrometastatic cell clusters (cell clusters of >10 cells). To determine angiogenic tumor growth in vivo histological samples may be taken at days 2, 3, 5, 7, and 10. Using anti-CD31 antibodies, tissues may be stained by immunoperoxidase, highlighting the vascular bed (endothelial surface). Anti-cytokeratin antibodies may be used with the same method for the purpose of highlighting the tumor cell clusters. Different chromogens (e.g. brown and red respectively) in order to study the relationship of vessels and tumor cell aggregates may be used.

Voxel-Based APC Radiopharmaceutical Radiation Therapy Planning

A tracer dose of I-124 labeled APC may be injected for planning PET/CT imaging. Because the same APC may be used for attaching the therapeutic nuclides, the biodistribution of I-124 in pre-treatment imaging would represent the distribution of therapeutic nuclides under therapy. Although it is reasonable to assume that the biodistribution of the APCs in each organ is relatively constant, their relative activities change following different time dependent courses. To take into account such kinetics, multiple PET studies may be performed. In one embodiment, voxel data from sequential I-124 APC PET images may be registered to the initial CT. A voxel-based dose kernel for the APC radionuclide of unit activity may be generated with Monte Carlo calculation. 3D maps of APC tissue residence at different times after injection are convolved with voxel dose kernel to compute a 3D dose map. Based on the dose prescriptions, the activity required to deliver the desired dose may be calculated.

Functions of the Treatment Planning System

Both CT images and PET images may be imported through DICOM transfers from the Syntegra software system associated with the Philips Gemini PET/CT. After the injection of ¹²⁴I labeled APC, the first PET/CT scans are automatically fused and can be viewed on the Syntegra system. Subsequent ¹²⁴I PET studies may also be transferred to Syntegra. For each of the subsequent PET scans, CT-PET fusion may be ensured by registering to the same laser markers tattooed on the patient. Software registration using the PET transmission images may be used to align the serial ¹²⁴I PET data sets. The kinetics of residence activity for the target (tumor) and major critical organs may be modeled. Each PET image series is a snapshot of the distribution of the APCs and the distribution within each organ also changes over time. Each voxel time-activity curve may be fitted to a multiexponential function and integrated to determine the accumulated activity and residence time.

Generate Unit Activity Voxel Dose Kernel

Because of the relatively short penetration of both α- and β-emitters, no sub-sampling may be required for dose calculation. The voxel dose kernel is the dose distribution resulting from a single voxel of a uniform radioactivity of 1 GBq in homogeneous water. It is calculated by using Monte-Carlo assuming spatially uniform activity over the volume of the source voxel.

Dose Rate Calculation for Static Radioactivity Distribution

If tissue heterogeneity is ignored, dose calculation can be approximated by a convolution of an invariant voxel dose kernel (isotropic) with the radioactivity distribution obtained from the PET scan (equation 1) where D(r) is the dose rate, A(r) is the activity distribution in a structure, and k(r) is the voxel dose kernel.

$$\begin{matrix}D ( r ) = A ( r ) ⊗ k ( r ) & ( 1 ) \\ D ( r ) = {\int }_{0}^{∞} {D}_{o} ( r ) \cdot h ( t ) d t & ( 2 )\end{matrix}$$

To account for the tissue heterogeneity, the voxel dose kernel is scaled according to the tissue density surrounding the source voxel. Based on the time dependent function for every structure, considering the kinetics, the decay, and the radioactivity loss, the dose may be integrated numerically over time, (equation 2) where D(r) is the accumulative dose distribution, Do(r) is the initial dose rate, h(t) is the time dependent function of the activity.

Calculate the Prescribed Therapeutic Activity Based on Dose Distribution

Where the physician desires to have a certain dose level cover all the tumors, the total activity required to be prescribed may also be calculated in accordance with the teachings of the instant invention. The final dose in Gy may be displayed on the CT images. Both 2D and 3D iso-dose surface display may be provided. A 2D and 3D plan summary and the total activity needed to be prescribed may be printed as part of the patient record.

Dose Verification after Administration of GAR

The same planning system in accordance with the invention may also be used to verify the dose delivered to the patient. By chelating low activities of I-124 with the therapeutic compound, the distribution of the radioactivity can be assessed with PET/CT. Following the same process as for pre-treatment planning, the administered doses at different times as well as the cumulative dose can be calculated.

By way of example, a subject may receive a single intravenous 0.5 mg dose of APC radiolabeled with 370 MBqs of I-124. A 10 mCi dose is estimated to permit more accurate biodistribution and dosimetric determinations (including imaging) with the lowest level of radioactive exposure to the subjects. Epinephrine, anti-histamines and corticosteroids may be available for use in the unlikely event of an immediate hypersensitivity reaction.

Serial I-124 PET total body imaging is performed, and pharmacokinetic studies as outlined.

| | Dose (% of predicted |
|---|---|
| | maximum organ |
| Cohort | tolerance) |
| 1 | 40 |
| 2 | 60 |
| 3 | 80 |
| 4 | 100 |

Whole body images (to include head, neck, chest, abdomen, pelvis, and proximal extremities) at 30 minutes, 2, 4, 24, 48, 96 hours and venous blood samples may be obtained at 5 and 30 minutes and at 1, 2, 4, 8, 12, and 24 hours after tracer administration. Prior to study and 24 hours later, blood and urine samples may be obtained for profile chemistries, complete blood counts, and urinalysis. Vital signs may be monitored at 0, 5, 15, 30 and 120 minutes and then at 12 and 24 hours after injection. Diagnostic polymer (APCDx) images may be evaluated for uptake of radioactivity in the target lesion(s). In addition, urine may be collected for 24 hours post-APCDx administration for use in dosimetry calculations.

Patients who are eligible for APCRx administration may first undergo a 18FDG-PET scan to provide additional imaging of target lesions. Then, within 7 days after the APCDx dose, the patient may receive an intravenous dose of APCRx depending on the stage of the dose escalation plan. Patients may undergo whole body I-124 PET imaging at approximately 1, 4.5, 24 and 48 hours following administration of APCRx and SPECT imaging at approximately 3.5 hours post-administration. In addition, urine and blood may be collected for 48 hours post-APCRx administration for use in dosimetry calculations.

Dose Estimation Using the MIRD Framework

Dosimetry for the APCDx and APCRx doses may be calculated from the whole body scans using the medical internal radiation dose (MIRD) approach. The dose delivered to tumor and normal organs by the therapeutic radiopharmaceutical may be estimated using a tracer administration of the compound labeled with iodine-124. The assumption is that the iodine-124 compound may have a similar biodistribution to the therapeutic compound and, because it is a positron emitter, its distribution over time can be measured using PET. Information about the source distribution can be combined with the known radiation characteristics (type of radiation, energy, half-life) of the therapeutic compound to estimate the dose that might be delivered to different organs. Internal dose estimates may be calculated in this way using the established framework developed by the Medical Internal Radiation Dose (MIRD) committee.

The high spatial resolution of PET and accurate attenuation correction means that both tumor volume and tumor activity concentration over time can be monitored. Serial patient I-124 PET studies performed over a period of 96 hours may be used to measure the residence time in the source regions. PET data may be acquired over the whole body and a flat scanner table may be used to aid reproducible patient positioning. All images may be calibrated in terms of absolute activity concentration and may be corrected for attenuation, scatter, randoms and radioactive decay of I-124. In addition to the PET images, transmission images may be acquired using a standard ¹³⁷Cs source for attenuation correction and to aid image registration. Venous blood samples may be taken over this same period to calculate the dose to blood and to measure the presence of any free I-124.

Image analysis may be performed within the Syntegra software environment. A mutual information registration algorithm, which is implemented within Syntegra, may be used to register the dynamic PET data. Regions-of-interest may be defined and applied to each of the I-124 PET images to obtain time-activity curves for different organs. A multi-exponential function may then be fit to these time-activity data and the resulting function may be integrated to determine the cumulative activity. The MIRD framework may be employed to determine the dose due to both beta and alpha radiation.

While the invention has been described in connection with specific embodiments thereof, it may be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

What is claimed:

1. An anti-angiogenic polymer conjugate (APC) for treatment of solid tumors, comprising: a water-soluble polymer backbone modified with a plurality of side chains, wherein at least two of said plurality of side chains harbor a chemical moiety capable of targeting a cell-surface protein of an endothelial cell present at an angiogenic site, wherein said cell-surface protein is an integrin.

2. The polymer conjugate according to claim 1 wherein the integrin is αᵥβ₃ integrin.

3. The polymer conjugate according to claim 1 wherein the chemical moiety is a ligand for the integrin.

4. The polymer conjugate according to claim 3 wherein the integrin is αᵥβ₃ integrin.

5. The polymer conjugate according to claim 3 wherein the ligand is RGD4C or RGDfK.

6. The polymer conjugate according to claim 5 wherein the RGD4C content comprises less than about 50 mole percent of the polymer conjugate.

7. The polymer conjugate according to claim 1 wherein greater than five side chains comprises a chemical moiety targeting the cell-surface protein of the endothelial cell at an angiogenic site.

8. The polymer conjugate according to claim 1 further comprising at least one side chain comprising a chelator capable of chelating a pharmaceutically acceptable radioactive label.

9. An anti-angiogenic polymer conjugate (APC) for treatment of solid tumors, comprising: a water-soluble polymer backbone modified with a plurality of side chains, wherein at least one of said plurality of side chains harbors a chemical moiety capable of targeting a cell-surface protein of an endothelial cell present at an angiogenic site, and wherein said cell-surface protein is an integrin; and
at least one side chain comprising a chelator, said chelator harboring a pharmaceutically acceptable alpha emitting radioactive label.

10. The polymer conjugate according to claim 9 wherein the alpha emitting radioisotope is ²¹³Bi or ²¹⁰Po.

11. An anti-angiogenic polymer conjugate (APC) for treatment of solid tumors, comprising: a water-soluble polymer backbone modified with a plurality of side chains, wherein at least one of said plurality of side chains harbors a chemical moiety capable of targeting a cell-surface protein of an endothelial cell present at an angiogenic site, and wherein said cell-surface protein is an integrin; and
at least one of the side chains comprising a chelator capable of chelating a pharmaceutically acceptable radioactive label.

12. The polymer conjugate according to claim 11 wherein the polymer backbone is electronegative.

13. The polymer conjugate according to claim 11 wherein the polymer backbone is N-(2-hydroxypropyl) methacrylamide (HPMA).

14. The polymer conjugate according to claim 11 wherein at least one of the plurality of side chains comprises a glycylglycine moiety.

15. The polymer conjugate according to claim 11 wherein the polymer backbone comprises a plurality of side chains comprising COOH groups.

16. The polymer conjugate according to claim 15 wherein the COOH groups comprise less than about 50 mole percent of the polymer conjugate.

17. The polymer conjugate according to claim 16 wherein the COOH groups comprise less than about 40 mole percent of the polymer conjugate.

18. The polymer conjugate according to claim 17 wherein the COOH groups comprise from about 10 mole percent to about 25 mole percent of the polymer conjugate.

19. The polymer conjugate according to claim 11 wherein radioactive label is an alpha, beta, gamma or positron emitting radioisotope.

20. The polymer conjugate according to claim 19 wherein the beta emitting radioisotope is ⁹⁰Y, ¹³¹I, ¹⁸⁸Re, or ¹⁷⁷Lu.

21. The polymer conjugate according to claim 11 wherein the radioactive label is selected from the group consisting of ¹²⁴I and ^{99m}Tc.

22. The polymer conjugate according to claim 11 wherein the chelator is selected from the group consisting of dipyridyllysine (“DPK”), m-hydroxybenzoic acid (“HBA”), 1,4,7,10-tetraazacyclododecane-1,4,7,10-tetraacetic acid (“DOTA”), and APMA-CHX-A″-DTPA.

23. The polymer conjugate according to claim 22 wherein the DOTA content comprises less than about 50 mole percent of the polymer conjugate.

24. The polymer conjugate according to claim 11 wherein the chelator content comprises less than about 50 mole percent of the polymer conjugate.

25. The polymer conjugate according to claim 24 wherein the chelator content comprises less than about 40 mole percent of the polymer conjugate.

26. The polymer conjugate according to claim 11 wherein said polymer conjugate has a molecular weight of less than about 45 kD.

27. A method of radiotherapy for the treatment of solid tumors comprising:
administering to a mammal harboring a solid tumor in need of said treatment, an effective dose of an anti-angiogenic polymer conjugate (APC), comprising: a water-soluble polymer backbone modified with a plurality of side chains, wherein at least one of said plurality of side chains harbors a chemical moiety that is directly coupled to the backbone with a chemical spacer and is capable of targeting a cell-surface protein of an endothelial cell present at an angiogenic site, wherein said cell-surface protein is an integrin, and
at least one of the side chains comprising a chelator capable of chelating a pharmaceutically acceptable radioactive label.

28. The method according to claim 32 wherein said polymer conjugate has a molecular weight of less than about 45 kD.

29. A method of localizing a radioactive nucleotide at the site of a solid tumor in a mammal, comprising: administering to said mammal an anti-angiogenic polymer conjugate (APC), comprising: a water-soluble polymer backbone modified with a plurality of side chains, wherein at least one of said plurality of side chains harbors a chemical moiety that is directly coupled to the backbone with a chemical spacer and is capable of targeting a cell-surface protein of an endothelial cell present at an angiogenic site, wherein said cell-surface protein is an integrin; and
at least one of the side chains comprising a chelator capable of chelating a pharmaceutically acceptable radioactive label.

30. A method of a determining a suitable radiotherapeutic regimen for treatment of a vascularized solid tumor in a mammal based on location and distribution of a tracer radioactive label, comprising:
(a) administering to the mammal a tracer dose of an APC according to claim 1; wherein the pharmaceutically acceptable radioactive label is a tracer label;
(b) determining the location and concentration of the tracer radioactive label within said mammal;
(c) calculating an amount of radioactivity required to deliver a therapeutic dose of a pharmaceutically acceptable radioactive label which is therapeutic.

31. The method of claim 30 wherein the tracer label is ¹²⁴I.

32. The method of claim 30 wherein the determination step (b) is performed by one or a combination of positron-emission tomography (PET) and computerized tomography (CT).

33. The method of claim 30 wherein the determination step (b) is performed over a predetermined period of time.

34. The method of claim 33 wherein the determination (b) further comprises modeling the kinetics of radioactivity in the tumor during the predetermined period of time.

35. The method of claim 30 wherein the tracer dose is administered intravenously.

36. The method of claim 30 wherein the tracer dose is from about 5 mCi to about 15 mCi.

37. The method of claim 36 wherein the tracer dose is about 10 mCi.

38. The method of claim 30 wherein the tracer dose of APC is from about 0.1 to about 1.0 mg.

39. The method of claim 38 wherein the tracer dose of APC is from about 0.5 mg.

40. The method of claim 30 wherein location and concentration of the tracer radioactive label the tumor of the mammal is determined.

41. The method of claim 30, further comprising administering to the mammal a dose of an APC according to claim 1 based on the amount calculated in step (c), wherein pharmaceutically acceptable radioactive label is therapeutic radioactive label.

42. A method of a determining a suitable radiotherapeutic regimen for treatment of a vascularized solid tumor in a mammal based on location and distribution of a tracer radioactive label, comprising:
(a) administering to the mammal a tracer dose of an APC according to claim 1, wherein the pharmaceutically acceptable radioactive label is a tracer label;
(b) determining the location and concentration of the tracer radioactive label within said mammal;
(c) calculating an amount of radioactivity required to deliver a therapeutic dose of a pharmaceutically acceptable therapeutic radioactive label.
(d) administering to the mammal a dose of an APC according to claim 1 based on the amount calculated in step (c), wherein the pharmaceutically acceptable radioactive label is therapeutic radioactive label.

43. The method of claim 42 wherein the location and concentration of the tracer label is determined at multiple locations within the mammal simultaneously.

44. The method of claim 42 wherein the determination step (b) is performed by one or a combination of positron-emission tomography (PET) and computerized tomography (CT).

45. An anti-angiogenic polymer conjugate (APC) for treatment of solid tumors, comprising: a water-soluble polymer backbone modified with a plurality of side chains, at least one side chain comprising a first chelator, said first chelator capable of harboring a first pharmaceutically acceptable radioactive label, and at least one side chain comprising a second chelator, said second chelator capable of harboring a second pharmaceutically acceptable radioactive label, in which the first and the second pharmaceutically acceptable radioactive labels decay by the emission of different energy particles.

46. The polymer conjugate according to claim 45 wherein the first and the second said pharmaceutically acceptable radioactive labels are alpha and beta emitters, respectively.

47. The polymer conjugate according to claim 45, further comprising at least one side chain comprising a third chelator, said third chelator capable of harboring a third pharmaceutically acceptable radioactive label, wherein the first, second, and third radioactive labels emit in different spectra.

48. The polymer conjugate according to claim 47, further comprising at least one side chain comprising a fourth chelator, said fourth chelator capable of harboring a fourth pharmaceutically acceptable radioactive label, wherein the first, second, third, and fourth radioactive labels emit in different spectra.

49. The polymer conjugate according to claim 48 wherein the first, second, third, and fourth pharmaceutically acceptable radioactive labels are independently one of alpha, beta, gamma, or positron emitters.

50. The polymer conjugate according to claim 45 wherein one of said pharmaceutically acceptable radioactive labels is ²¹⁰Po.

51. The polymer conjugate according to claim 45 further comprising at least one side chain comprising a chemical moiety targeting cell-surface proteins of endothelial cells at an angiogenic site.

52. The polymer conjugate according to claim 51 wherein the cell-surface protein is an integrin.

53. A method of treating a cancer patient in need of radiotherapy comprising: (a) providing a desired radiation emission profile that is tailored to a type and/or stage of cancer a patient is suffering from; (b) providing a plurality of anti-angiogenic polymer conjugate (APC) populations, each population harboring a particular radionuclide, the collective radiation emission profile of said plurality of APC populations substantially mimicking said desired radiation emission profile; (c) administering an effective amount of said plurality of APC populations to the cancer patient;
wherein said APC comprises a water-soluble polymer backbone modified with a plurality of side chains, wherein at least one of said plurality of side chains harbors a chemical moiety capable of targeting a cell-surface protein of an endothelial cell present at an angiogenic site, and wherein said cell-surface protein is an integrin; and
at least one of the side chains comprising a chelator capable of chelating a pharmaceutically acceptable radioactive label.

54. A method for calculating the dose of radiation for treatment of a mammal in need of radiation therapy comprising: (a) administering to the mammal a tracer radionuclide, (b) generating a plurality of images of the distribution and residence of the tracer radionuclide at multiple loci throughout the mammal at multiple time points following said administration, and (c) determining a dose of radiation based on the results of step (b).

55. The method of claim 54 wherein the generating step (b) is performed by one or a combination of positron-emission tomography (PET) and computerized tomography (CT).
